(19) United States
(12) Patent Application Publication
(10) Pub. No.: US 2026/0233022 A1
(43) Pub. Date: Aug. 13, 2026

(54) BIOPHOTONIC ENERGY HARVESTING FOR IMPLANTABLE DEVICES

(71) Applicant: Medtronic, Inc., Minneapolis, MN (US)

(72) Inventor: Tyler W. Stigen, Brooklyn Park, MN (US)

(21) Appl. No.: 19/531,921

(22) Filed: Feb. 6, 2026

Related U.S. Application Data
(60) Provisional application No. 63/755,952, filed on Feb. 7, 2025.

(51) Int. Cl. A61N 5/06

(52) U.S. Cl. CPC A61N 5/0601; A61N 5/062

(57) ABSTRACT

Systems and methods for biophotonic energy harvesting for implantable medical devices are provided. In some embodiments, an implantable medical device includes a housing configured to be implanted in a body of a patient. The implantable medical device can further include an energy harvesting mechanism carried by the housing. The energy harvesting mechanism can be configured to receive light from a light source located entirely within the body of the patient and to produce electrical energy from the received light. The implantable medical device can further include electrical circuitry coupled to the energy harvesting mechanism. The electrical circuitry can be configured to deliver at least some of the electrical energy to a device component carried by the housing to power the device component.

### RELATED APPLICATIONS

This application claims the benefit of U.S. Provisional Application Ser. No. 63/755,952, filed Feb. 7, 2025, the entire contents of each of which are incorporated herein by reference.

### TECHNICAL FIELD

The present technology generally relates to medical devices, and in particular, to biophotonic energy harvesting for implantable medical devices.

### BACKGROUND

Various types of implantable medical devices have been developed for monitoring or treating one or more conditions of a patient. For example, a cardiac monitor can measure signals reflecting a patient's heart activity, and a pacemaker can deliver electrical stimulation to the heart. These types of medical devices may be sized to be implanted entirely within or adjacent to the heart, and may have integrated electrodes rather than external leads. Conventionally, primary cell or rechargeable batteries are used to power these and other types of implantable medical devices. However, the relatively small size of these devices limits the types of power sources that can be incorporated into the device. Smaller power sources may have lower power capacity, which can limit the functionality and longevity of the device.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1 is a conceptual diagram illustrating an example medical system configured to monitor a patient, in accordance with embodiments of the present technology.

FIG. 2A is a perspective diagram of an example of an implantable medical device for monitoring a patient, in accordance with embodiments of the present technology.

FIG. 2B is a functional schematic diagram of an implantable medical device, in accordance with embodiments of the present technology.

FIG. 3 is a perspective diagram of an implantable medical device, in accordance with embodiments of the present technology.

FIG. 4 is a perspective view of a pacing device configured in accordance with embodiments of the present technology.

FIG. 5 is a side view of another pacing device configured in accordance with embodiments of the present technology.

FIG. 6 is a schematic block diagram illustrating electronic components of a pacing device configured in accordance with embodiments of the present technology.

FIG. 7A is a conceptual cross-sectional diagram of an implantable medical device including an energy harvesting mechanism, in accordance with embodiments of the present technology.

FIG. 7B is a conceptual cross-sectional diagram of an implantable medical device including an energy harvesting mechanism, in accordance with embodiments of the present technology.

FIG. 7C is a conceptual cross-sectional diagram of an implantable medical device including an energy harvesting mechanism, in accordance with embodiments of the present technology.

FIG. 8 is a conceptual cross-sectional diagram of an implantable medical device including an energy harvesting mechanism, in accordance with embodiments of the present technology.

FIG. 9 is a flow diagram illustrating a method for energy harvesting in an implantable medical device, in accordance with embodiments of the present technology.

### DETAILED DESCRIPTION

The present technology relates to systems, devices, and methods for biophotonic energy harvesting in implantable medical devices. In some embodiments, for example, an implantable medical device includes a housing configured to be implanted in a body of a patient. For instance, the implantable medical device can be positioned within or adjacent to the patient's heart. The implantable medical device can further include an energy harvesting mechanism carried by the housing. The energy harvesting mechanism can be configured to receive light from a light source located entirely within the body of the patient and to produce electrical energy from the received light. The light source can be a biological source. In some embodiments, the light source includes a tissue of the patient. Alternatively or in combination, the light source can include a bioreactor disposed in the housing, where the bioreactor includes a light-emitting species. The implantable medical device can further include electrical circuitry coupled to the energy harvesting mechanism, where the electrical circuitry is configured to deliver at least some of the electrical energy to a device component (e.g., a processor, sensor, electrode) carried by the housing to power the device component. For instance, the device component can be a sensing electrode, and the at least some of the electrical energy can power the sensing electrode to measure electrical activity within the patient (e.g., electrical activity of the patient's heart).

The present technology can provide numerous advantages compared to conventional approaches for powering implantable medical devices. For instance, the energy harvesting mechanisms herein can produce electrical energy from light received within a body of a patient, thereby extending the lifetime of the implantable medical device by allowing for intrinsic power generation, e.g., for recharging a power source in situ within the patient's body or for directly powering device functionalities. In some embodiments, the light is received from naturally-occurring light sources in the body, such as living tissues configured to emit light, either endogenously or via genetic modification. In other embodiments, light-emitting species may be provided within the device. By leveraging light sources within the patient's body, the present technology can provide sustainable energy that prolongs the device's longevity. Further, the use of light sources within the body can reduce or eliminate the need for external charging solutions and/or complex device circuitry that may be required for other energy harvesting modalities such as modalities based on mechanical energy, heat energy, electrostatic energy, ambient radiofrequency energy, etc.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

As used herein, the terms “vertical,” “lateral,” “upper,” and “lower” can refer to relative directions or positions of features of the embodiments disclosed herein in view of the orientation shown in the Figures. For example, “upper” or “uppermost” can refer to a feature positioned closer to the top of a page than another feature. These terms, however, should be construed broadly to include embodiments having other orientations, such as inverted or inclined orientations where top/bottom, over/under, above/below, up/down, and left/right can be interchanged depending on the orientation.

The headings provided herein are for convenience only and do not interpret the scope or meaning of the claimed present technology. Embodiments under any one heading may be used in conjunction with embodiments under any other heading.

### I. Implantable Medical Devices and Systems

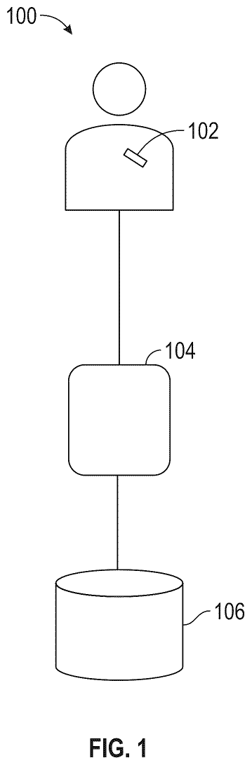

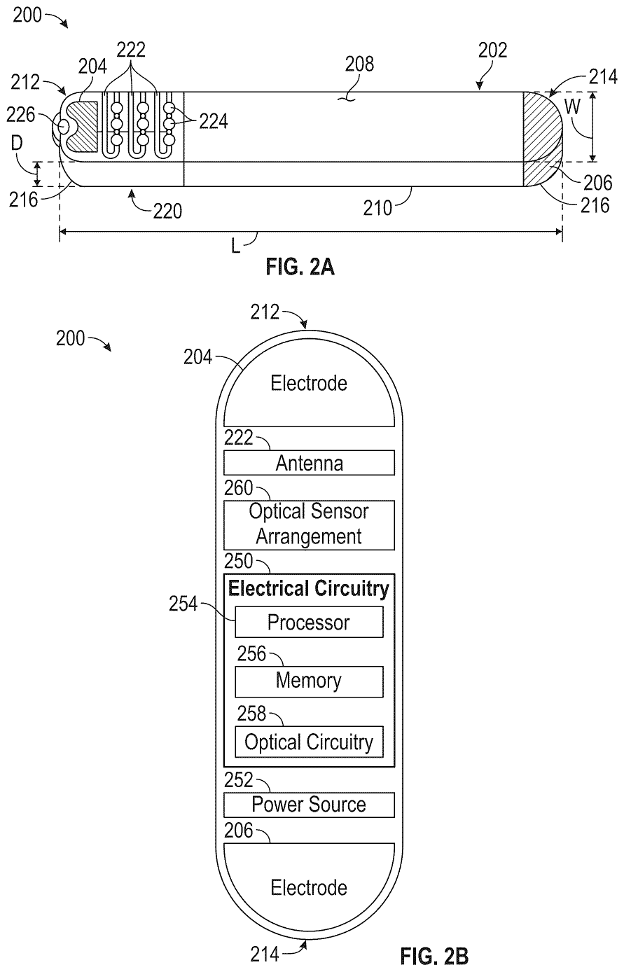

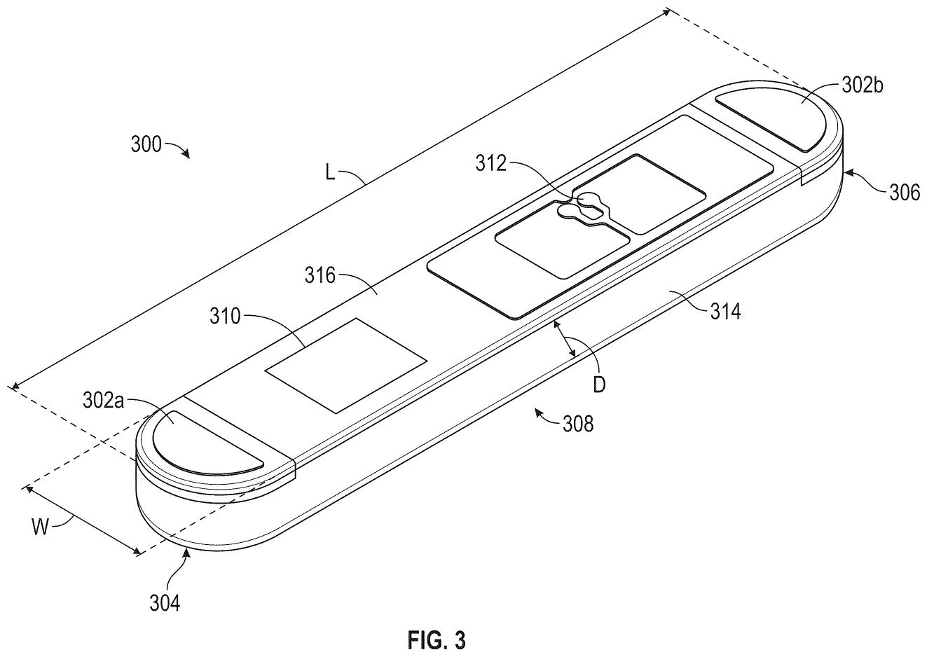

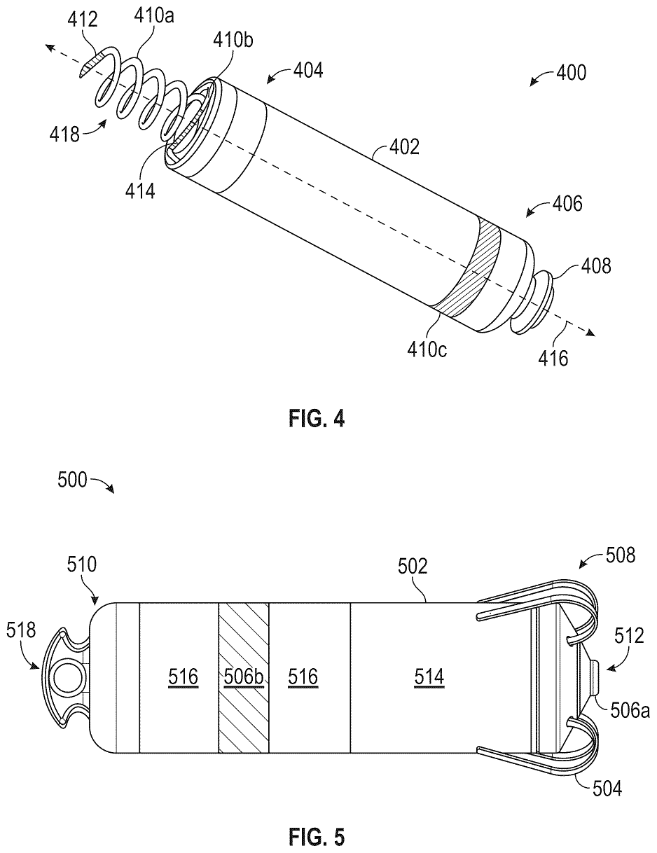

FIGS. 1-6 provide a general overview of implantable medical devices and systems configured in accordance with embodiments of the present technology. Specifically, FIG. 1 illustrates a cardiac medical system, FIGS. 2A-3 illustrate implantable medical devices for monitoring a patient, and FIGS. 4-6 illustrate implantable medical devices for pacing a patient's heart. Any of the features of the embodiments of FIGS. 1-6 can be combined with each other and/or with any of the other embodiments described herein (e.g., the embodiments described in Section II below).

Referring first to FIG. 1, a cardiac medical system 100 may include an implantable medical device 102, which may include a communication module for communicating with a programmer 104. The programmer 104 may include a user interface that presents information to and receives input from a user. In some embodiments, the programmer 104 may include, for example, a suitable computing device such as a tablet, a smartphone, desktop computer, laptop computer, and/or the like. It should be noted that the user may also interact with programmer remotely via a networked computing device. As further shown in FIG. 1, in some embodiments, the implantable medical device 102 and/or the programmer 104 may be configured to transfer and/or receive information (e.g., cardiac data, such as intracardiac electrogram (EGM) data and/or cardiac episode-related information derived from the EGM data) to and/or from a secondary memory storage device 106, such as over a wired or wireless network.

A user, such as a physician, technician, surgeon, electrophysiologist, other clinician, or patient, interacts with the programmer to communicate with implantable medical device 102. For example, the user may interact with the programmer to retrieve physiological or diagnostic information from the implantable medical device 102. A user may also interact with the programmer to program the implantable medical device 102, e.g., select values for operational parameters of the implantable medical device 102. For example, the user may use the programmer to retrieve information from the implantable medical device 102 regarding the rhythm of a patient heart, trends therein over time, or arrhythmic episodes. In some embodiments, alerts regarding device status (e.g., health state) and/or regarding type(s) of cardiac episode(s) detection may be provided to the patient or a clinician through the programmer 104, though they may be provided in any suitable manner (e.g., personal smartphone, other computing device, pushed through to an electronic medical record, etc.). The implantable medical device 102 and the programmer may communicate via wireless communication using any techniques known in the art.

In some embodiments, the implantable medical device 102 can be placed subcutaneously in a patient near or over the patient's heart. For example, in some embodiments the implantable medical device 102 can be placed in a subcutaneous pocket located over an intercostal space (e.g., over the 4th intercostal space), and positioned at a desirable angle and/or displacement relative to the patient's sternum (e.g., between about 0 and 45 degrees relative to the sternum, about 2 cm from the left edge of the sternum). Once inserted, the implantable medical device 102 may go through suitable setup and/or calibration processes.

In some embodiments, the implantable medical device 102 is implanted outside of a thoracic cavity of a patient (e.g., subcutaneously in a pectoral location). The implantable medical device 102 may be positioned near the sternum near or just below the level of the heart of the patient, e.g., at least partially within the cardiac silhouette. In some embodiments, the implantable medical device 102 includes a plurality of electrodes and is configured to sense a cardiac electrogram (EGM) via the plurality of electrodes, as well as other physiological signals and/or parameters via an optical sensor arrangement. In some embodiments, the implantable medical device 102 takes the form of an insertable cardiac monitor (ICM) such as the LINQ™ or LINQ II™ ICM, or other ICM similar to, e.g., a version or modification of the LINQ™ or LINQ II™ ICM. Representative examples of ICMs are described below in connection with FIGS. 2A-3.

Alternatively or in combination, the implantable medical device 102 can take the form of a leadless pacemaker. For instance, the implantable medical device 102 can be a pacing device configured to be implanted entirely within a heart chamber, such as entirely within the right atrium (RA), entirely within the right ventricle (RV), entirely within the left atrium (LA), or entirely within the left ventricle (LV). The device 102 can be implanted at any of a variety of locations to sense and/or deliver therapy to any chamber or chambers of the heart. For example, the device 102 can be a right atrial intracardiac pacemaker that is implanted in the RA of the patient's heart in a target implant region (e.g., the triangle of Koch). The target implant region can lie between the bundle of His and the coronary sinus, and/or can be adjacent to the tricuspid valve. In other embodiments, the device 102 can instead be configured as a right ventricular intracardiac pacemaker that is implanted in the RV of the heart, with the target implant region lying along the endocardial wall at or near the apex of the RV. In some examples, the implantable medical device 102 takes the form of a leadless intracardiac pacemaker such as the Micra™ AV2 or Micra™ AV2 leadless pacemaker, or other leadless pacemaker similar to, e.g., a version or modification of the Micra™ AV2 or Micra™ AV2. Representative examples of leadless intracardiac pacemakers are described below in connection with FIGS. 4-6.

FIG. 2A is a perspective diagram of an example of an implantable medical device 200 (also referred to herein as a “cardiac monitoring device”) for monitoring a patient, in accordance with embodiments of the present technology. The implantable medical device 200 is an example of an implantable medical device 102 that may be used in the system 100 of FIG. 1. In the example shown in FIG. 2A, the implantable medical device 200 may be embodied as a monitoring device having a housing 202, a first (e.g., proximal) electrode 204, and a second (e.g., distal) electrode 206. The housing 202 may further include a first major surface 208, a second major surface 210, a first (e.g., proximal) end 212, and a second (e.g., distal) end 214. The housing 202 encloses electrical circuitry 250 and power source 252 (shown in FIG. 2B) located inside the implantable medical device 200 and protects the circuitry contained therein from body fluids. Electrical feedthroughs provide electrical connection of the electrodes 204 and 206.

In some embodiments such as that shown in FIG. 2A, the implantable medical device 200 is defined by a length L, a width W, and a thickness or depth D. The implantable medical device 200 may be in the form of an elongated rectangular prism wherein the length L is much larger than the width W, which in turn is larger than the depth D. In some embodiments, the geometry of the implantable medical device 200 (for example, a width W greater than the depth D) may be selected to allow the implantable medical device 200 to be inserted under the skin of the patient using a minimally invasive procedure and to remain in the desired orientation during insert. For example, the device shown in FIG. 2A may include radial asymmetries (notably, the rectangular shape) along the longitudinal axis that maintains the device in the proper orientation following insertion. For example, in some embodiments the spacing between the proximal electrode 204 and the distal electrode 206 may range from 30 millimeters (mm) to 55 mm, 35 mm to 55 mm, and from 40 mm to 55 mm and may be any range or individual spacing from 25 mm to 60 mm. In addition, the length L of the implantable medical device 200 may range from 30 mm to about 70 mm. In other embodiments, the length L may range from 40 mm to 60 mm, 45 mm to 60 mm and may be any length or range of lengths between about 30 mm and about 70 mm. In addition, the width W of the first major surface 208 may range from 3 mm to 10 mm and may be any single or range of widths between 3 mm and 10 mm. In some embodiments, the thickness or depth D of the implantable medical device 200 may range from 2 mm to 9 mm. For example, the depth D of the implantable medical device 200 may range from 2 mm to 5 mm and may be any single or range of depths from 2 mm to 9 mm. In addition, the implantable medical device 200, according to an example embodiment, has a geometry and size designed for ease of implant and patient comfort. Embodiments of the implantable medical device 200 described in this disclosure may have a volume of three cubic centimeters (cm) or less, 1.5 cubic cm or less or any volume between three and 1.5 cubic cm.

In the example shown in FIG. 2A, once inserted within the patient, the first major surface 208 faces outward, toward the skin of the patient while the second major surface 210 is located opposite the first major surface 208. In addition, in the example shown in FIG. 2A, the proximal end 212 and the distal end 214 are rounded to reduce discomfort and irritation to surrounding tissue once inserted under the skin of the patient. The implantable medical device 200, including instruments and methods for inserting the device 200, is described, for example, in U.S. Patent Publication No. 2014/0276928, incorporated herein by reference in its entirety.

In some embodiments, the proximal electrode 204 and the distal electrode 206 are used to sense cardiac signals for determining a cardiac event (e.g., bradycardia or asystole event) such as EGM signals, intra-thoracically or extra-thoracically, which may be sub-muscularly or subcutaneously. EGM signals may be stored in a memory of the implantable medical device 200, and EGM data may be transmitted via integrated antenna 222 to another medical device, which may be another implantable medical device or an external device.

In the example embodiment shown in FIG. 2A, the proximal electrode 204 is in close proximity to the proximal end 212 and the distal electrode 206 is in close proximity to the distal end 214. In this embodiment, the distal electrode 206 is not limited to a flattened, outward-facing surface, but may extend from the first major surface 208 around rounded edges 216 and onto the second major surface 210 so that the distal electrode 206 has a three-dimensional curved configuration. In the example embodiment shown in FIG. 2A, the proximal electrode 204 is located on the first major surface 208 and is substantially flat and outward facing. However, in other embodiments, the proximal electrode 204 may utilize the three-dimensional curved configuration similar to that of distal electrode 206, providing a three-dimensional proximal electrode (not shown in this embodiment). Additionally or alternatively, in other embodiments, the distal electrode 206 may utilize a substantially flat, outward-facing electrode located on the first major surface 208 similar to that shown with respect to the proximal electrode 204. The various electrode configurations allow for configurations in which the proximal electrode 204 and the distal electrode 206 are located on both the first major surface 208 and the second major surface 210. In other configurations, such as that shown in FIG. 2A, only one of the proximal electrode 204 and the distal electrode 206 is located on both the major surfaces 208 and 210. In still other configurations, both the proximal electrode 204 and the distal electrode 206 are located on one of the first major surface 208 or the second major surface 210 (e.g., the proximal electrode 204 located on the first major surface 208 while the distal electrode 206 is located on the second major surface 210). In some embodiments, the implantable medical device 200 may include electrodes on both the first major surface 208 and the second major surface 210 at or near the proximal and distal ends of the device, such that a total of at least four electrodes are included on the implantable medical device 200. The electrodes 204 and 206 may be formed of a plurality of different types of biocompatible conductive material (e.g. stainless steel, titanium, platinum, iridium, or alloys thereof), and/or may utilize one or more coatings such as titanium nitride or fractal titanium nitride.

In the example shown in FIG. 2A, the proximal end 212 includes a header assembly 220 that includes one or more of the proximal electrode 204, the integrated antenna 222, anti-migration projections 224, and/or suture hole 326. The integrated antenna 222 may be located on the same major surface (e.g., the first major surface 208) as the proximal electrode 204 and may also be included as part of the header assembly 220. The integrated antenna 222 allows the implantable medical device 200 to transmit and/or receive data. In some embodiments, the integrated antenna 222 may be formed on the opposite major surface as the proximal electrode 204, or may be incorporated within the housing 202 of the implantable medical device 200. In the example embodiment shown in FIG. 2A, the anti-migration projections 224 are located adjacent to the integrated antenna 222 and protrude away from the first major surface 208 to prevent longitudinal movement of the device 200, though may be arranged on any suitable surface of the implantable medical device 200. In the example embodiment shown in FIG. 2A, the anti-migration projections 224 include a plurality (e.g., nine) of small bumps or protrusions extending away from the first major surface 208; however, the anti-migration projections 224 may additionally or alternatively be located on the opposite major surface as the proximal electrode 204 and/or the integrated antenna 222. As shown in FIG. 2A, the suture hole 226, which may be used to help secure the implantable medical device 200 in the patient to prevent movement following insertion of the implantable medical device 200, may be located adjacent to the proximal electrode 204, though one or more suture holes 226 may additionally or alternatively be located in any other suitable location. In some embodiments, the header assembly 220 is a molded header assembly made from a polymeric or plastic material, which may be integrated or separable from the main portion of the implantable medical device 200.

FIG. 2B is a functional schematic diagram of an implantable medical device, such as the implantable medical device 200 as shown in FIG. 2A, in accordance with embodiments of the present technology. Although the reference numbers refer to the implantable medical device 200, it should be understood that other implantable medical devices described herein (e.g., the implantable medical device 300 of FIG. 3) can include one or more components similar to that described below. The implantable medical device 200 includes housing 202, proximal electrode 204 located at proximal end 212, distal electrode 206 located at distal end 214, integrated antenna 222, electrical circuitry 250, and power source 252. In some embodiments, the implantable medical device 200 includes an optical sensor arrangement 260 comprising an emitter set of one or more optical light emitters and a detector set of one or more optical light detectors. The optical sensor arrangement 260 may, for example, be configured to provide a photoplethysmography (PPG) signal using the emitter and detector sets.

The optical sensor arrangement 260 can be configured to sense through one or more surfaces of the implantable medical device 200 (e.g., first major surface 208, second major surface 210, a surface of the header assembly 220). In some embodiments, one or more portions of the one or more surfaces comprise a material that is optically transparent to at least some wavelengths of light. For example, the one or more portions of the one or more surfaces through which the optical sensor arrangement 260 senses can be transparent to a red wavelength, transparent to a green wavelength, and/or transparent to an infrared wavelength. In some embodiments, the one or more portions of the one or more surfaces are optically transparent to visible light (e.g., electromagnetic radiation with a wavelength from approximately 380 nm to approximately 780 nm). The orientation of the optical sensor arrangement 260 with respect to the patient is based on the implantation of the implantable medical device 200. For example, in some embodiments (e.g., embodiments wherein the optical sensor arrangement 260 senses through the first major surface 208), the optical sensor arrangement 260 is directed away from a center of the patient (e.g., oriented distally) when the implantable medical device 200 is implanted within the patient, and thus is exposed to a maximal amount of ambient light.

Fidelity of ambient light sensing can correlate to factors external to the optical sensor arrangement 260, such as physical activity of a patient and the environment surrounding the patient. Sensing from the optical sensor arrangement 260 can be affected when the patient moves vigorously and/or when then patient is in an environment of intense and/or rapidly varying ambient light. In some embodiments, the optical sensor arrangement 260 is configured to sense in response to sensor data (e.g., motion data, optical data). For example, in some embodiments, a motion sensor (e.g., an accelerometer) senses physical activity of the patient and while the patient is below a first motion threshold as sensed by the motion sensor (e.g., when the patient is resting, when the patient remains still), electrical circuitry 250 directs (e.g., via optical circuitry 258) the optical sensor arrangement 260 to sense. In some embodiments, the motion sensor senses the patient is above a second motion threshold (e.g., the patient is moving vigorously) and the electrical circuitry 250 directs the optical sensor arrangement 260 not to sense. In some embodiments, the optical sensor arrangement 260 senses for a first period of time to determine whether optical data is suitable for the optical sensor arrangement 260 to sense at a second period of time (e.g., immediately after, continuously until another condition is met) or whether the optical sensor arrangement 260 should sense at a third period of time (e.g., after a duration of time such as 1 minute).

The electrical circuitry 250 may be coupled to the optical sensor arrangement 260 to sense optical signals (e.g., via the optical circuitry 258) corresponding to PPG and/or ambient light. The electrical circuitry 250 may be coupled to the proximal electrode 204 and the distal electrode 206 to sense cardiac signals and monitor events (e.g., arrythmia, etc.). The electrical circuitry 250 is also connected to transmit and receive communications via the integrated antenna 222. The power source 252 provides power to the electrical circuitry 250, as well as to any other components that require power. The power source 252 may include one or more energy storage devices, such as one or more rechargeable or non-rechargeable batteries. The implantable medical device 200 as shown in FIGS. 2A and 2B may be a monitoring-only device. However, in other examples, implantable medical device 200 may further provide therapy delivery capabilities.

The electrical circuitry 250 is configured to receive multiple signal types. For example, the electrical circuitry 250 can receive raw EGM signals monitored by the proximal electrode 204 and the distal electrode 206 and/or PPG signals monitored by the optical sensor arrangement 260. The electrical circuitry 250 may also include components/modules for converting a raw signal (e.g., EGM, PPG) to a processed signal that can be analyzed to detect sense events. Although not shown, the electrical circuitry 250 may include any discrete and/or integrated electronic circuit components that implement analog and/or digital circuits capable of producing the functions described for analyzing EGM and/or PPG signals to detect/verify bradycardia and/or asystole events. For example, the electrical circuitry 250 may include analog circuits, e.g., pre-amplification circuits, filtering circuits, and/or other analog signal conditioning circuits. The modules may also include digital circuits, e.g., digital filters, combinational or sequential logic circuits, state machines, integrated circuits, one or more processors 254 (shared, dedicated, or group) that executes one or more software or firmware programs, memory devices 256, or any other suitable components or combination thereof that provide the described functionality.

In some embodiments, the electrical circuitry 250 may include a sensing unit for monitoring signals detected (e.g., by the proximal electrode 204 and the distal electrode 206, by the optical sensor arrangement 260), and at least one sensing channel that utilizes an algorithm for identifying events in the signal (e.g., the EGM signal, the PPG signal). For example, sensed events (e.g., R-waves) are utilized to detect one or more cardiac episodes. In some embodiments, the electrical circuitry 250 includes the one or more processors 254 configured to receive information regarding the sensed events and implements one or more algorithms for determining whether a particular one or more events have occurred. In addition, the analog voltage signals received from the electrodes 204 and 206 and/or the optical sensor arrangement 260 may be passed to analog-to-digital (A/D) converters (ADC) included in the electrical circuitry 250, and stored in the memory unit 256 included as part of the electrical circuitry 250 for subsequent analysis with firmware executed by the processor(s) 254 included as part of the electrical circuitry 250.

The electrical circuitry 250 may control functions of the implantable medical device 200 and process signals received from the electrodes 204 and 206 (e.g., EGM signals) and/or the optical sensor arrangement 260 (e.g., optical signals) according to programmed signal analysis routines or algorithms. The implantable medical device 200 may include the optical circuitry 258 to facilitate optical signal detection, processing, and/or control. The implantable medical device 200 may include other optional sensors (not shown) for monitoring physiological signals, such as an activity sensor, pressure sensor, oxygen sensor, accelerometer, and/or other sensor used to monitor a patient. These may also be provided to the electrical circuitry 250 for processing.

The electrical circuitry 250 may similarly control monitoring time intervals and sampling rates according to a particular clinical application. In addition, electrical circuitry may include state machines or other sequential logic circuitry to control device functions and need not be implemented exclusively as a microprocessor.

The electrical circuitry 250 communicates with the integrated antenna 222 or other communication to transmit electrical signal data, e.g. EGM signal data, stored in memory or received from the electrical circuitry 250 in real time. The antenna 222 may be configured to transmit and receive communication signals via inductive coupling, electromagnetic coupling, tissue conductance, Near Field Communication (NFC), Radio Frequency Identification (RFID), BLUETOOTH®, WiFi, or other proprietary or non-proprietary wireless telemetry communication schemes.

The electrical circuitry 250 may include a communication module including the integrated antenna 222, so as to enable the implantable medical device 200 to communicate with one or more external devices located external to the device 200.

FIG. 3 is a perspective diagram of an implantable medical device 300, in accordance with embodiments of the present technology. The implantable medical device 300 may be a leadless, subcutaneously implantable monitoring device including a proximal electrode 302a located at proximal end 304, a distal electrode 302b located at distal end 306 (collectively “electrodes 302”), a housing 308, electrical circuitry (not shown), an optical sensor arrangement 310 (comprising, for example optical sensor(s)), an integrated antenna 312, and a power source (not shown). In particular, the electrical circuitry is coupled to proximal electrode 302a and distal electrode 302b to sense cardiac signals and monitor events. The electrical circuitry may also be connected to transmit and receive communications via the integrated antenna 312. The power source can provide power to electrical circuitry, as well as to any other components that require power. The power source may include one or more energy storage devices, such as one or more rechargeable or non-rechargeable batteries. In some examples, the electrical circuitry includes processing circuitry and a storage device, such as memory, the memory being operatively coupled to the processing circuitry and configured to store data and/or instructions.

In the example shown in FIG. 3, the electrical circuitry may receive raw EGM or EMG (electromyography) signals monitored by the proximal electrode 302a and distal electrode 302b and raw optical signals monitored by the optical sensor arrangement 310. The electrical circuitry may include components/modules for converting the raw EGM signal to a processed EGM signal that can be analyzed to detect sense events and for converting the raw optical signals to calibrated processed optical signal(s) that can be analyzed to detect sense events. Although not shown, the electrical circuitry may include any discrete and/or integrated electronic circuit components that implement analog and/or digital circuits capable of producing the functions described for analyzing optical signal(s) to determine a health condition status of a patient. For example, the electrical circuitry may include analog circuits, e.g., pre-amplification circuits, filtering circuits, and/or other analog signal conditioning circuits. The modules may also include digital circuits, e.g., digital filters, combinational or sequential logic circuits, state machines, integrated circuits, a processor (shared, dedicated, or group) that executes one or more software or firmware programs, memory devices, or any other suitable components or combination thereof that provide the described functionality.

In one example, the electrical circuitry includes a sensing unit for monitoring the EGM signal detected by the respective proximal electrode 302a and the distal electrode 302b, as well as light signals received by the optical sensor arrangement 310, respectively. In one example, the electrical circuitry includes processing circuitry that is utilized to receive information regarding sensed events and to implement one or more algorithms for determining a health condition status of a patient. In addition, the analog voltage signals received from the electrodes 302 may be passed to analog-to-digital (A/D) converters included in the electrical circuitry and stored in the memory unit included as part of the electrical circuitry for subsequent analysis with firmware executed by the processor included as part of the electrical circuitry.

Some embodiments of the implantable medical device 300 include a container 314 and an insulative cover 316. In some examples, the insulative cover 316 may include an optical window. In some examples, the optical window may be formed of the same material as insulative cover 316. In some examples, the optical window may be a portion of insulative cover 316. The proximal electrode 302a and the distal electrode 302b may be formed or placed on an outer surface of the cover 316. The electrical circuitry may be formed or placed on an inner surface of the cover 316, or within the container 314. In some examples, the antenna 312 is formed or placed on the inner surface of the cover 316. In other examples, the antenna 312 is formed or placed on the outer surface of the cover 316, or the antenna 312 may be formed or placed at least partially on the inner surface and partially on the outer surface of the cover 316. In some examples, the insulative cover 316 may be positioned over the open container 314 such that the container 314 and the cover 316 form the housing 308 and enclose the electrical circuitry (and in some cases the antenna 312) to protect the circuitries from fluids such as body fluids. For example, the housing 308 may be a hermetically-sealed housing configured for subcutaneous implantation within a patient, wherein at least the power source, the memory, and the processing circuitry are within the hermetically-sealed case, and in some examples, the optical sensor arrangement 310 is also within the hermetically-sealed case.

The electrical circuitry may be formed on the inner side of the insulative cover 316, such as by using flip-chip or wire bond integrated circuit packaging technology. The insulative cover 316 may be flipped onto the container 314. When flipped and placed onto the container 314, the components of implantable medical device 300 formed on the inner side of the insulative cover 316 may be positioned in a gap defined by the container 314. The electrodes 302 and the antenna 312 (when placed or formed on the outer surface of the cover 316) may be electrically connected to sensing circuitry and communication circuitry, respectively, e.g., through one or more vias formed through the insulative cover 316. The insulative cover 316 may be formed of sapphire (i.e., corundum), glass, and/or any other suitable insulating material. The container 314 may be formed from any suitable material configured to house electrical circuitry, to support and mate with the cover 316 to isolate electrical circuitry from contact with tissue and/or fluids of a patient, and to be implantable within the patient. In some examples, the container 314 may house the power source (e.g., a battery). In some examples, the container 314 may also be electrically conductive. For example, the container 314 may be formed from titanium or any other suitable material (e.g., a biocompatible material). The electrodes 302 may be formed from any of stainless steel, titanium, platinum, iridium, or alloys thereof. In addition, the electrodes 302 may be coated with a material such as titanium nitride or fractal titanium nitride, although other suitable materials and coatings for such electrodes may be used.

In some embodiments, the implantable medical device 300 is defined by a length L, a width W and thickness or depth D and is in the form of an elongated rectangular prism wherein the length L is much larger than the width W, which in turn is larger than the depth D, as illustrated in FIG. 3. In one example, the geometry of the implantable medical device 300 (e.g., in particular the width W being greater than the depth D) is selected to allow the implantable medical device 300 to be inserted under the skin of the patient using a minimally invasive procedure and to remain in the desired orientation during insert. For example, the implantable medical device 300 may include a radial asymmetry (notably, a rectangular shape) along the longitudinal axis that maintains the device in the proper orientation following insertion. In one example, the spacing between the proximal electrode 302a and the distal electrode 302b may range from 30 millimeters (mm) to 55 mm, 35 mm to 55 mm, and from 40 mm to 55 mm and may be any range or individual spacing from 25 mm to 60 mm. In another example, the spacing between the proximal electrode 302a and the distal electrode 302b may range from 15 mm to 30 mm, 17 mm to 28 mm, and from 20 mm to 28 mm and may be any range or individual spacing from 12 mm to 30 mm. In addition, the length L of the implantable medical device 300 may range from 30 mm to about 70 mm. In other embodiments, the length L may range from 40 mm to 60 mm, 45 mm to 60 mm and may be any length or range of lengths between about 30 mm and about 70 mm. In some examples, the length L of the implantable medical device 300 may range from 15 mm to about 35 mm, or from 20 mm to 30 mm, 22 mm to 30 mm and may be any length or range of lengths between about 15 mm and about 35 mm. In addition, the width W of a major surface of the implantable medical device 300, e.g., the insulative cover 316 in the example shown in FIG. 3, may range from 3 mm to 10 mm and may be any single or range of widths between 3 mm and 10 mm, or may range from 1.5 mm to 5 mm and may be any single or range of width between 1.5 mm and 5 mm. The thickness or depth D of the implantable medical device 300 may range from 2 mm to 9 mm, or from 1.5 mm to 4.5 mm. In other embodiments, the depth D of the implantable medical device 300 may range from 2 mm to 5 mm and may be any single or range of depths from 2 mm to 9 mm, or may range from 1 mm to 2.5 mm and may be any single or range of depths from 1 mm to 4.5 mm. In addition, the implantable medical device 300, according to an example of the present technology, may have a geometry and size designed for ease of implant and patient comfort. Examples of the implantable medical device 300 described in this disclosure may have a volume of 3 cubic cm or less, 1.5 cubic cm or less or any volume between 3 and 1.5 cubic cm, or may have a volume of 1.5 cubic cm or less, 0.75 cubic cm or less or any volume between 1.5 and 0.75 cubic cm.

FIG. 4 is a perspective view of a pacing device 400 configured in accordance with embodiments of the present technology. The pacing device 400 is an example of an implantable medical device 102 that may be used in the system 100 of FIG. 1. The device 400 is configured to be implanted within a chamber of a heart of the patient to monitor activity of the heart and/or provide electrical therapy (e.g., pacing therapy) to the heart. The device 400 includes a housing 402 having a size and form factor that allows the device 400 to be entirely implanted within a single chamber of the patient's heart. In the illustrated embodiment, the housing 402 has an elongate shape (e.g., a generally cylindrical shape, a generally prismatic shape) extending between a distal end 404 and proximal end 406. The housing 402 can define a hermetically sealed internal cavity for housing the electronic components of the device 400. The housing 402 can also include an attachment mechanism 408 (e.g., at the proximal end 406) configured to temporarily engage with a delivery tool during implantation and/or extraction of the device 400.

The housing 402 can be formed partially or entirely from a conductive material, such as titanium or titanium alloy, stainless steel, MP35N (a non-magnetic nickel-cobalt-chromium-molybdenum alloy), a platinum alloy, or other biocompatible metal or metal alloy, or other suitable conductive material. Alternatively or in combination, the housing 402 can be formed partially or entirely from a nonconductive (e.g., insulative) material, such as ceramic, glass, sapphire, silicone, polyurethane, epoxy, acetyl co-polymer plastics, polyether ether ketone (PEEK), a liquid crystal polymer, other biocompatible polymer, or other suitable nonconductive material.

The device 400 can include a plurality of electrodes 410a-410c configured to sense electrical activity of the heart and/or deliver electrical stimulation to the heart. In the illustrated embodiment, for example, the device 400 includes a first electrode 410a and a second electrode 410b at or proximate to the distal end 404 of the housing 402, and a third electrode 410c on the housing 402. The first and second electrodes 410a, 410b can be configured as cathode electrodes that directly contact cardiac tissue, e.g., a distal end of the first electrode 410a can be configured to rest within a ventricular myocardium of the patient, and the second electrode 410b can be configured to contact an atrial endocardium of the patient. The third electrode 410c can be configured as an anode and/or return electrode that does not directly contact cardiac tissue.

As shown in FIG. 4, the first electrode 410a can be an elongate structure that extends from the distal end 404 of the housing 402 to penetrate through the wall tissue of a first heart chamber (e.g., the chamber in which the device 400 is implanted) into wall tissue of a second, different heart chamber. For example, in some embodiments, the device 400 is implanted in the RA with the distal end 404 oriented toward the LV (e.g., similar to the arrangement of the device 102 in FIG. 1), and the first electrode 410a extends through the wall tissue of the RA and into the wall tissue of the LV. In the illustrated embodiment, the first electrode 410a is configured as a coil (e.g., a helical and/or spiral coil), while in other embodiments, the first electrode 410a can have a different form factor (e.g., an elongate dart, barb, tine, or other tissue penetrating element). The first electrode 410a can include a proximal end that is coupled to the distal end 404 of the housing 402, and a free distal end that is not attached to the housing 402. The distal end of the first electrode 410a can have a conical, hemi-spherical, or slanted edge distal tip with a narrow tip diameter (e.g., less than 1 mm) for penetrating into and through tissue layers. In some embodiments, the distal end of the first electrode 410a can have a sharpened or angular tip, and/or sharpened or beveled edges, but the degree of sharpness can be constrained to avoid a cutting action that could lead to lateral displacement of the distal end of the first electrode 410a and undesired tissue trauma.

The second electrode 410b can be a structure that extends from the distal end 404 of the housing 402 to contact the wall tissue of the first heart chamber without penetrating the wall tissue. The second electrode 410b can be located proximal to the first electrode 410a. The second electrode 410b can be configured as a coil (e.g., a partial helical and/or spiral coil that does not form a full turn), loop, button, pad, or any other suitable form factor. The second electrode 410b can include a proximal end that is coupled to the distal end 404 of the housing 402, and a distal end that may or may not be coupled to the housing 402. In some embodiments, the second electrode 410b is configured to flexibly maintain contact with wall tissue of the heart chamber in which the device 400 is implanted, (e.g., the RA endocardium), despite variations in the tissue surface and/or in the distance between the distal end 404 of the housing 402 and the tissue surface, which may occur as the wall tissue moves during the cardiac cycle. Accordingly, the second electrode 410b can be flexible and/or have spring-like properties, e.g., the second electrode 410b can have a spring bias that urges at least a portion of the second electrode 410b away from the distal end 404 of the housing 402 and toward the wall tissue of the heart chamber to maintain consistent contact.

The first and second electrodes 410a, 410b can each be formed of an electrically conductive material, such as titanium, platinum, iridium, tantalum, or alloys thereof. The first electrode 410a can include one or more insulative coatings (e.g., parylene, polyurethane, silicone, epoxy) that reduce the electrically conductive surface area of the first electrode 410a to define a first electrically active region 412 (e.g., at or near the distal end of the first electrode 410a). The second electrode 410b can include one or more insulative coatings (e.g., parylene, polyurethane, silicone, epoxy) that reduce the electrically conductive surface area of the second electrode 410b to define a second electrically active region 414 (e.g., at an intermediate region between the proximal and distal ends of the second electrode 410b). This approach can increase the electrical impedance of the first and second electrodes 410a, 410b, and thereby reduce the current delivered during a pacing pulse, which can conserve the power used by the device 400. In some embodiments, the first and second electrodes 410a, 410b include an electrically conductive material coating (e.g., TiN) on the first and second electrically active regions 412, 414, respectively, to define the active regions. The first and second electrodes 410a, 410b can be made of the same materials, or can be made of different materials.

All, substantially all, or a portion of the housing 402 can serve as a third electrode 410c (e.g., an anode and/or return electrode) during pacing and/or sensing. In some embodiments, the third electrode 410c partially or fully circumscribes a portion of the housing 402 at or near the proximal end 406. Although FIG. 4 illustrates the third electrode 410c as a singular band, in other embodiments, the third electrode 410c can include multiple segments spaced a distance apart along a longitudinal axis 416 of the housing 402 and/or around a perimeter of the housing 402. Additionally, the third electrode 410c can also be located at other positions along the housing 402, e.g., located at or near the distal end 404 or at other positions along the longitudinal axis 416.

In embodiments where the housing 402 is formed from a conductive material, one or more portions of the housing 402 can be electrically insulated by a nonconductive material, such as a coating of parylene, polyurethane, silicone, epoxy or other biocompatible polymer, or other suitable material. For the portions of the housing 402 without the nonconductive material, one or more discrete areas of the housing 402 with conductive material can be exposed to define the third electrode 410c. In embodiments where the housing 402 is formed from a nonconductive material, a conductive material can be applied to one or more discrete areas of the housing 402 to form the third electrode 410c. Optionally, the third electrode 410c can be a discrete component (e.g., a ring electrode) that is coupled to the housing 402.

The electrodes 410a-410c can be used to sense electrical activity of one or more heart chambers and/or to deliver electrical stimulation to one or more heart chambers. For example, the first electrode 410a can be paired with the second electrode 410b or the third electrode 410c to for sensing ventricular signals and delivering ventricular pacing pulses. As another example, the second electrode 410b can be paired with the first electrode 410a or the third electrode 410c for sensing atrial signals and delivering pacing pulses to the atrial myocardium. In a further example, the third electrode 410c can be paired at different times with both the first electrode 410a and the second electrode 410b for either ventricular or atrial functionality, respectively. As yet another example, the first electrode 410a and the second electrode 410b can be paired with each other with different polarities for atrial and ventricular functionality.

In some embodiments, the second electrode 410b is configured as an atrial cathode electrode for delivering pacing pulses to the atrial tissue at a target implant region in combination with the third electrode 410c. The second electrode 410b and the third electrode 410c can also be used to sense atrial P-waves for use in controlling atrial pacing pulses (e.g., delivered in the absence of a sensed P-wave) and for controlling atrial-synchronized ventricular pacing pulses delivered using the first electrode 410a as a cathode and the third electrode 410c as the return anode. The configuration of the electrodes 410a-410c illustrated in FIG. 4 allows the device 400 to sense cardiac signals from and/or deliver cardiac pacing to one or more chambers of the heart. For example, the present technology can facilitate the delivery of A-V synchronous pacing using a single device 400 implanted within a single heart chamber (e.g., the RA).

The device 400 can include a fixation mechanism 418 configured to fix the device 400 to cardiac tissue at a target implant region (e.g., the triangle of Koch). In the illustrated embodiment, the first electrode 410a and/or second electrode 410b at the distal end 404 of the housing 402 can serve as the fixation mechanism 418. In other embodiments, the fixation mechanism 418 can be a different component than the first electrode 410a and/or the second electrode 410b, such one or more separate barbs, tines, coils, darts, etc.

FIG. 5 is a side view of another pacing device 500 configured in accordance with embodiments of the present technology. The pacing device 500 is an example of an implantable medical device 102 that may be used in the system 100 of FIG. 1. The device 500 is configured to be implanted within a chamber of a heart of a patient to monitor activity of the heart and/or to provide electrical therapy to the heart. In the embodiment shown in FIG. 5, the device 500 includes a housing 502, a plurality of fixation tines 504, a first electrode 506a, and a second electrode 506b.

The housing 502 can have a size and form factor that allows the device 500 to be entirely implanted within a chamber of a heart of a patient. For example, as shown in FIG. 5, the housing 502 has a generally cylindrical (e.g., pill-shaped or capsule-shaped), elongate form factor extending between a distal end 508 and a proximal end 510. The housing 502 contains electronic components of the device 500, and can be hermetically or near-hermetically sealed to prevent fluid ingress into the housing 502. The materials used to form the housing 502 can include any of the conductive and nonconductive materials described above with respect to FIG. 4.

The device 500 can include a fixation mechanism configured to fix the device 500 to cardiac tissue at a target implant region (e.g., the endocardial wall near the apex of the RV). In the illustrated embodiment, the device 500 includes a plurality of fixation tines 504 extending from the distal end 508 of the housing 502 and configured to engage with cardiac tissue to secure the housing 502 at a fixed position within the chamber of the heart. The fixation tines 504 can be configured to anchor the housing 502 to the cardiac tissue such that the device 500 moves along with the cardiac tissue during cardiac contractions. The device 500 can include any suitable number of fixation tines 504, such as one, two, three, four, five, or more fixation tines 504. The fixation tines 504 can be fabricated from any suitable material, such as a shape memory material (e.g., Nitinol). Alternatively or in combination, the device 500 can be fixed to cardiac tissue using other types of fixation mechanisms, such as, but not limited to, barbs, coils, darts, and the like.

Optionally, the device 500 can include an attachment mechanism configured to temporarily couple the device 500 to a delivery tool, e.g., for delivery and/or extraction of the device 500. In the illustrated embodiment, for example, the proximal end 510 includes a flange 518 that defines an opening. The flange 518 can be attached to a tether (e.g., by threading the tether through the opening) that extends through an elongate shaft (e.g., a catheter) to implant or extract the device 500.

In some embodiments, the device 500 is configured to sense electrical activity of the heart and/or deliver electrical stimulation to the heart via the first electrode 506a and second electrode 506b (collectively, “electrodes 506”). The first electrode 506a can serve as a cathode configured to electrically contact cardiac tissue and deliver pacing pulses thereto, and the second electrode 506b can serve as an anode and/or a return electrode. Optionally, the device 500 can be equipped with multiple cathode electrodes. Such multiple cathode electrodes can be configured to electrically contact and deliver pacing pulses to cardiac tissue of a single heart chamber, or cardiac tissue of multiple heart chambers. In some such embodiments, the multiple cathode electrodes are configured to electrically contact and deliver pacing pulses to cardiac tissue of different heart chambers. For example, one cathode electrode can be configured to electrically contact and deliver pacing pulses to atrial tissue, and another cathode electrode may be configured to electrically contact and deliver pacing pulses to ventricular tissue.

The electrodes 506 can be configured in many different ways. For example, one or both of the electrodes 506 can be discrete components that are mechanically coupled to the housing 502. As another example, one or both of the electrodes 506 can be defined by an outer portion of the housing 502 that is electrically conductive. The electrodes 506 can be electrically isolated from each other. In some embodiments, a portion of the housing 502 is covered by or formed from an insulative material to isolate the electrodes 506 from each other and/or to provide a desired size and shape for one or both of the electrodes 506. The electrodes 506 can be electrically coupled to at least some of the internal electronic components of the device 500 within the housing 502 (e.g., sensing circuitry, electrical stimulation circuitry, or both).

In the illustrated embodiment, the first electrode 506a is located at the distal end 508 of the housing 502. The first electrode 506a may be referred to as a tip electrode, and the fixation tines 504 can be configured to anchor the device 500 to cardiac tissue such that the first electrode 506a maintains contact with the cardiac tissue. In some examples, the housing 502 includes an end cap 512 at the distal end 508, and the end cap 512 includes a feedthrough assembly to electrically couple the first electrode 506a to the electronics within the housing 502, while electrically isolating the first electrode 506a from the remaining portions of the housing 502, e.g., including the second electrode 506b and/or other conductive portions of the housing 502

The second electrode 506b can be located on the housing 502 away from (e.g., proximal to) the first electrode 506a. As shown in FIG. 5, the housing 502 includes a first portion 514 and a second portion 516, with the first portion 514 being located proximal to the end cap 512, and the second portion 516 being located proximal to the first portion 514. The second portion 516 can optionally define at least part of a power source case that houses a power source (e.g., a battery) of the pacing device 500. In some embodiments, the second electrode 506b is located on the second portion 516, while in other embodiments, the second electrode 506b is located on the first portion 514.

In some embodiments, the second electrode 506b is a conductive portion of the housing 502 (e.g., an annular portion of the housing 502 that is made partially or entirely from a conductive material). Additionally or alternatively, the second electrode 506b can be a conductive material that is coated onto the material of the housing 502, or a discrete component (e.g., a ring electrode) that is coupled to the housing 502. The remaining portions of the housing 502 can include or be coated with an insulative material so that the second electrode 506b is electrically isolated from the rest of the housing 502 and/or from the first electrodes 506a.

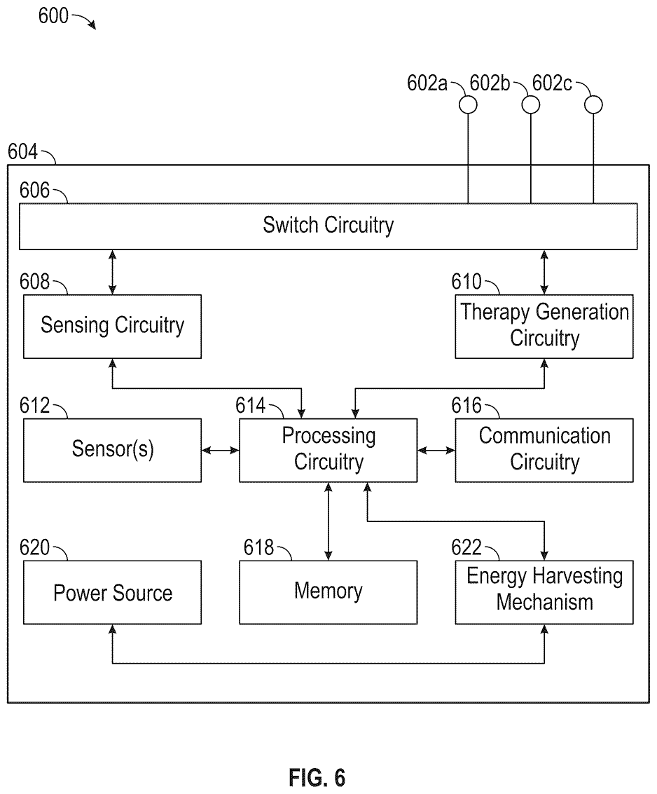

FIG. 6 is a schematic block diagram illustrating electronic components of a pacing device 600 configured in accordance with embodiments of the present technology. Any of the electronic components shown in FIG. 6 can be incorporated into any of the embodiments of implantable medical devices described herein, such as the implantable medical device 102 of FIG. 1, the device 400 of FIG. 4, or the device 500 of FIG. 5.

As shown in FIG. 6, the device 600 includes a plurality of electrodes 602a-602c that are electrically coupled to components within a housing 604 of the device 600. Although the device 600 is illustrated and described herein as having three electrodes 602a-602c (e.g., similar to the device 400 of FIG. 4), in other embodiments, the device 600 can be modified to include a different number of electrodes, such as two electrodes (e.g., similar to the device 500 of FIG. 5) or any other suitable number of electrodes.

At least some of the electrodes 602a-602c can be configured to contact tissue of one or more heart chambers, as described elsewhere herein. For example, as discussed above with respect to FIG. 4, the first electrode 602a can be configured to electrically contact and deliver electrical signals to tissue of a first heart chamber (e.g., ventricular tissue), and the second electrode 602b can be configured to electrically contact and deliver electrical signals to tissue of a second, different heart chamber (e.g., atrial tissue). The third electrode 602c can be an anode and/or return electrode that does not electrically contact heart tissue. Optionally, either the first electrode 602a or the second electrode 602b can be omitted, or the device 600 can include additional electrodes that electrically contact and deliver electrical signals to tissue of a heart chamber (e.g., the first heart chamber, the second heart chamber, or another heart chamber).

The device 600 includes a plurality of electronic components within the housing 604, such as switch circuitry 606, sensing circuitry 608, therapy generation circuitry 610, one or more sensors 612, processing circuitry 614, communication circuitry 616, memory 618, and/or a power source 620. The various circuitry can be or include programmable or fixed function circuitry configured to perform the operations described herein. One or more of the components of the device 600 shown in FIG. 6 can be part of an electronics assembly. For example, one or more of the switch circuitry 606, sensing circuitry 608, therapy generation circuitry 610, sensor(s) 612, processing circuitry 614, communication circuitry 616, and/or memory 618 can be mounted on a circuit board of an electronics assembly of the device 600.

The switch circuitry 606 can include one or more switches (e.g., a switch matrix, switch arrays, or other collection of switches), multiplexers, transistors, and/or other electrical circuitry. The switch circuitry 606 can selectively couple one or more of the electrodes 602a-602c to other components of the device 600 (e.g., the sensing circuitry 608 and/or the therapy generation circuitry 610). The subset of the electrodes 602a-602c to be used can depend on the particular operation of the device 600 that is being performed, such as whether the device 600 is sensing or delivering therapy, the locations of the heart being monitored or treated, etc. In some embodiments, the processing circuitry 614 determines which subset of the electrodes 602a-602c should be used for a particular operation, and controls the switch circuitry 606 to selectively couple those electrodes to the appropriate components of the device 600.

The sensing circuitry 608 can monitor signals from at least one of electrodes 602a-602c to monitor electrical activity of the heart, impedance, and/or other electrical phenomena. Sensing can be performed to determine heart rates and/or heart rate variability, and/or to detect ventricular dyssynchrony, arrhythmias (e.g., tachyarrhythmias), and/or other electrical signals. The sensing circuitry 608 can include filters, amplifiers, analog-to-digital converters, and/or other circuitry configured to sense cardiac electrical signals via one or more of the electrodes 602a-602c.

In some embodiments, the switch circuitry 606 as controlled by the processing circuitry 614 selectively couples the sensing circuitry 608 to selected combinations of the electrodes 602a-602c, e.g., to selectively sense the electrical activity of one or more chambers of the heart. For example, the switch circuitry 606 can couple each of the first electrode 602a and the second electrode 602b (in combination with the third electrode 602c) to respective sensing channels provided by the sensing circuitry 608 to sense electrical signals from the cardiac tissues in electrical contact with the first electrode 602a (e.g., ventricular tissue) and the second electrodes 602b (e.g., atrial tissue), respectively. In some embodiments, the sensing circuitry 608 is configured to detect events, (e.g., depolarizations) within the cardiac electrical signals, and to provide indications thereof to the processing circuitry 614. In this manner, the processing circuitry 614 can determine the timing of atrial and/or ventricular depolarizations, and can control the delivery of cardiac pacing (e.g., AV synchronized cardiac pacing) based thereon.

The therapy generation circuitry 610 can generate electrical stimulation signals, such as cardiac pacing pulses. The therapy generation circuitry 610 can be electrically coupled to one or more of the electrodes 602a-602c to deliver pulses to a portion of cardiac muscle within the heart via one or more of the electrodes 602a-602c. In some embodiments, the therapy generation circuitry 610 delivers pacing stimulation in the form of electrical pulses. The therapy generation circuitry 610 can include charging circuitry, and one or more charge storage devices (e.g., capacitors). Optionally, the therapy generation circuitry 610 can include switches and/or other circuitry to control when the charge storage devices are discharged to the electrodes 602a-602c.

The switch circuitry 606 as controlled by the processing circuitry 614 can direct electrical stimulation signals from the therapy generation circuitry 610 to a selected combination of the electrodes 602a-602c having selected polarities, e.g., to selectively deliver pacing pulses to the RA, RV, LV, and/or the interventricular septum of the heart. For example, in order to pace one or both of the ventricles, the switch circuitry 606 can electrically couple the first electrode 602a (e.g., which contacts wall tissue of a ventricle or the intraventricular septum) to the therapy generation circuitry 610 as a cathode, and to one or both of the second electrode 602b or the third electrode 602c to the therapy generation circuitry 610 as an anode. As another example, in order to pace the RA, the switch circuitry 606 can couple the second electrode 602b (e.g., which contacts the RA endocardium) to the therapy generation circuitry 610 as a cathode, and to one or both of the first electrode 602a or the third electrode 602c to the therapy generation circuitry 610 as an anode.

The processing circuitry 614 can include one or more processors, such as a microprocessor, a controller, a digital signal processor (DSP), an application specific integrated circuit (ASIC), a field-programmable gate array (FPGA), or equivalent discrete or analog logic circuitry. In some embodiments, the processing circuitry 614 can include multiple components, such as any combination of one or more microprocessors, controllers, DSPs, ASICs, and/or FPGAs, as well as other discrete or integrated logic circuitry. The functions attributed to the processing circuitry 614 herein may be embodied as software, firmware, hardware, or any combination thereof.

The processing circuitry 614 can control the therapy generation circuitry 610 to deliver stimulation therapy to a patient's heart according to therapy parameters, which can be stored in the memory 618. For example, the processing circuitry 614 can control the therapy generation circuitry 610 to deliver electrical pulses with the amplitudes, pulse widths, rates, frequencies, and/or electrode polarities specified by the therapy parameters. In this manner, the therapy generation circuitry 610 can deliver pacing pulses to the heart via one or more of the electrodes 602a-602c. The device 600 can use any combination of the electrodes 602a-602c to deliver therapy and/or detect electrical signals from the patient.

The memory 618 (e.g., a data storage device or other non-transitory medium) can store computer-readable instructions that, when executed by the processing circuitry 614, cause the device 600 to perform the various operations described herein. The memory 618 can include any volatile, non-volatile, magnetic, optical, or electrical media, such as a random-access memory (RAM), read only memory (ROM), non-volatile RAM (NVRAM), electrically-erasable programmable ROM (EEPROM), flash memory, or any other digital or analog media.

The sensor(s) 612 can include one or more sensing elements that transduce patient physiological activity to an electrical signal to sense values of a respective patient parameter. Sensor(s) 612 can include one or more motion sensors, optical sensors, chemical sensors, temperature sensors, pressure sensors, and/or any other types of sensors. The sensor(s) 612 can output patient parameter values to the processing circuitry 614 that can be used as feedback to control sensing and/or delivery of therapy by the device 600.

For example, the sensor(s) 612 can include at least one motion sensor, such as one or more inertial measurement units (IMUs), accelerometers, gyroscopes, electrical or magnetic field sensors, and/or other devices capable of detecting motion and/or the position of the device 600. The motion of the device 600 detected by the motion sensor may be indicative of cardiac events (e.g., paced activation of the ventricles), blood flow through the heart, patient posture, patient activity, and/or noise. The processing circuitry 614 can control and/or monitor the motion data produced by the motion sensor to identify one or more features of the cardiac contraction within the signal (e.g., on a beat-by-beat basis or otherwise) to facilitate delivery of therapy (e.g., delivery of ventricular pacing pulses in an atrial-synchronized manner). Optionally, the processing circuitry 614 can use the motion data to detect a current activity level of the patient, which can be used for rate-responsive pacing of the patient's heart.

The communication circuitry 616 is configured to allow the device 600 to wirelessly communicate with another device, such as a device external to the patient's body (e.g., the programmer 104 of FIG. 1) and/or another device under the control of the processing circuitry 614. For instance, the processing circuitry 614 can receive updates to operational parameters from the other device, and/or can provide collected data, (e.g., sensed heart activity and/or other patient parameters) to the other device via the communication circuitry 616. The communication circuitry 616 can use radiofrequency (RF) communication techniques (e.g., via an antenna) and/or any other suitable communication modality.

The power source 620 delivers operating power to various components of the device 600. The power source 620 can include one or more batteries, each of which can independently be rechargeable or non-rechargeable. Recharging of the power source 620 can be accomplished using an energy harvesting mechanism 622 of the device 600. Additional details of energy harvesting mechanisms and associated methods are provided in Section II below.

The components of the device 600 illustrated in FIG. 6 can be modified in many different ways. For example, any of the components shown in FIG. 6 can be combined with each other, e.g., the switch circuitry 606 can be incorporated into the sensing circuitry 608 and/or the therapy generation circuitry 610. Any of the components shown in FIG. 6 can be divided into smaller subcomponents. Some of the components in FIG. 6 are optional and may be omitted (e.g., the switch circuitry 606 and/or sensor(s) 612). The device 600 can also include additional components not shown in FIG. 6. For example, the device 600 can include power management circuitry coupled to the power source 620 to allow the processing circuitry 614 to monitor the status of the power source 620 (e.g., charge level, charging rate, net power into and/or out of the power source 620, remaining battery life).

The components of the devices shown in FIGS. 1-6 represent functionality that can be included in any of the devices of the present technology. The components illustrated in FIGS. 1-6 can include any discrete and/or integrated electronic circuit components that implement analog and/or digital circuits capable of producing the functions attributed to the components herein. For example, the components can include analog circuits, such as amplification circuits, filtering circuits, and/or other signal conditioning circuits. The components can also include digital circuits, such as combinational or sequential logic circuits, memory devices, and the like. The functions attributed to the components of FIGS. 1-6 may be embodied as one or more processors, hardware, firmware, software, or any combination thereof. The depiction of different features as separate blocks in FIGS. 1-6 is intended to highlight different functional aspects, and does not necessarily imply that such components must be realized by separate hardware or software components. Rather, functionality associated with one or more components may be performed by separate hardware or software components, or integrated within common or separate hardware or software components.

### II. Devices and Methods for Biophotonic Energy Harvesting

In some embodiments, the present technology provides implantable medical devices that include an energy harvesting mechanism. As noted previously, the power capacity of a power source of an implantable medical device may be limited due to size constraints, such as if the device is implanted within a small space within the patient's body (e.g., subcutaneously, within a single heart chamber) and/or to avoid the device interfering with normal physiological function, as well as safety considerations. To prolong the usable life of such implantable medical devices, an energy harvesting mechanism can be used to generate energy in situ to recharge the power source.

In some embodiments, an energy harvesting mechanism of an implantable medical device is configured to produce electrical energy from light received from light sources within a body of a patient (e.g., a light source that is located entirely within the patient's body and/or is not coupled to any components external to the patient's body). For instance, the energy harvesting mechanism can include a photovoltaic material, and the photovoltaic material can convert the received light into electrical energy. As will be described further herein, the light can be received from naturally-occurring light sources (e.g., cells that are native to the patient's body), as well as non-naturally-occurring light sources (e.g., cells that are genetically modified and/or are not native to the patient's body). The light source may be a biological light source, such as a cell, tissue, microorganism, etc. Light produced by a biological light source may be referred to herein as “biophotons.” The electrical energy produced by the energy harvesting mechanism can be used to charge a power source of the implantable medical device and/or directly power one or more device functions.

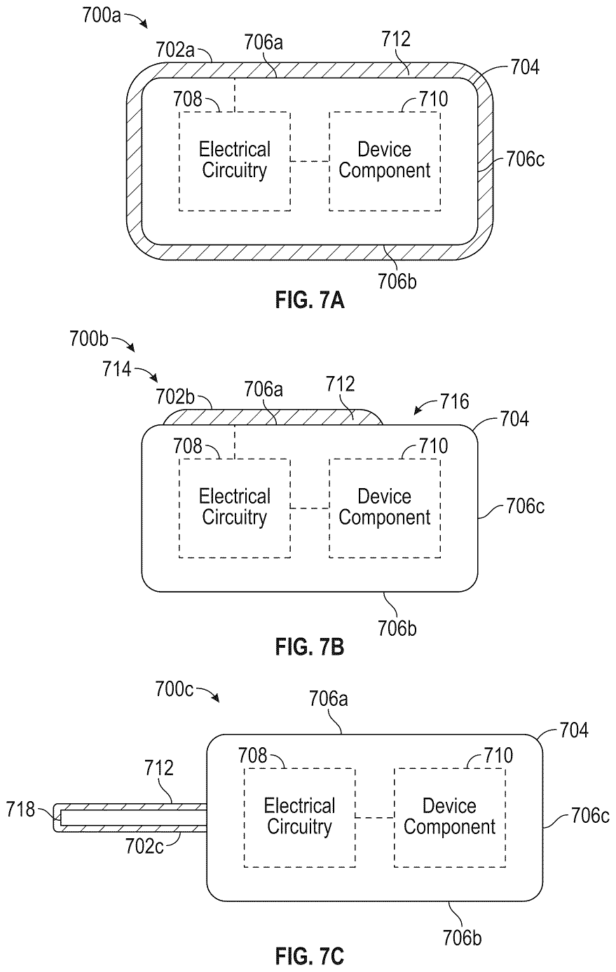

FIG. 7A is a conceptual cross-sectional diagram of an implantable medical device 700a including an energy harvesting mechanism 702a, in accordance with embodiments of the present technology. The implantable medical device 700a is an example of a medical device 102 that may be used in the system 100 of FIG. 1. The implantable medical device 700a can be an insertable cardiac monitor for monitoring activity of a patient's heart (e.g., as described in connection with FIGS. 2A-3) and/or a pacing device configured to provide electrical stimulation to the patient's heart (e.g., as described in connection with FIGS. 4-6). Accordingly, the implantable medical device 700a may additionally or alternatively include any of the components of the devices described herein, such as any of the devices described in connection with FIGS. 1-6. Moreover, in other embodiments, the implantable medical device 700a need not be used for monitoring and/or stimulating the heart, and can be used for other therapeutic applications and/or elsewhere in the body.

The implantable medical device 700a can include a housing 704 carrying (e.g., coupled directly or indirectly to, containing, or otherwise supporting) the energy harvesting mechanism 702a. The housing 704 can have one or more external surfaces, such as an upper surface 706a, a lower surface 706b opposite the upper surface 706a, and a plurality of side surfaces 706c. The housing 704 is illustrated conceptually and may correspond (e.g., have the same or similar geometries) to device housings of any of the implantable medical devices described herein. For instance, the housing 704 may correspond to the housing 202 of the implantable medical device 200 of FIG. 2, where the upper surface 706a corresponds to the first major surface 208, the lower surface 706b corresponds to the second major surface 210, etc.

The housing 704 can define an interior cavity containing electrical circuitry 708 and one or more device components 710. The device components 710 can include any of the components described herein, such as a power source, processor, memory, one or more electrodes, one or more sensors, sensing circuitry, therapy generation circuitry, switch circuitry, communication circuitry, etc. Optionally, one or more of the device components 710 may be located on an external surface of the housing 704. As an example, the device components 710 may include one or more electrodes located on the upper surface 706a.

In some embodiments, the energy harvesting mechanism 702a includes one or more photovoltaic cells including a photovoltaic material 712 that converts light energy into electrical energy via the photovoltaic effect. For instance, the photovoltaic material 712 can be disposed on at least a portion of the housing 704, such as on any of the surfaces 706 of the housing 704, and the photovoltaic material 712 may be configured to receive light from light sources external to the housing 704 of the implantable medical device 700a and to produce electrical energy from the received light.

The photovoltaic material 712 can be any of a plurality of materials configured to produce the photovoltaic effect. The photovoltaic effect refers to a phenomenon in which light absorption by a material causes excitation of electrons within the material, providing sufficient energy for the electrons to vacate their atoms, and thereby creating a separation between the electrons and electron-holes (e.g., electron-hole pairs). This separation of charge creates an electric potential and a current flow, which can be redirected and/or stored. In some embodiments, the photovoltaic material 712 includes a semiconductor such as silicon. The silicon can be a monocrystalline silicon or a polycrystalline silicon, or a combination thereof. Alternatively or in combination, the photovoltaic material 712 can include other semiconductors, such as gallium arsenide, cadmium telluride (CdTe), copper indium gallium selenide (CIGS), etc. Other photovoltaic materials 712 are possible, such as perovskite photovoltaics (e.g., methylammonium lead triiodide (MAPbI₃)), organic photovoltaics, etc. Further, additional optical materials may be used to enhance the absorption of the photovoltaic material 712. For instance, a mirror or lens may be used to concentrate and/or direct light toward the photovoltaic material 712.

The photovoltaic material 712 can have any suitable geometry. For instance, the photovoltaic material 712 may be deposited as a thin film, coating, layer, quantum dot, etc., on the housing 704. The thickness of the photovoltaic material 712 can be no more than 20 nm, 50 nm, 100 nm, 500 nm, 1 μm, 20 μm, 50 μm, 100 μm, or 500 μm. In the illustrated embodiment of FIG. 7A, the photovoltaic material 712 is disposed over the entirety of the housing 704 (e.g., over the upper surface 706a, lower surface 706b, and side surfaces 706c). However, in other embodiments, the photovoltaic material 712 may alternatively or additionally be configured differently as will be described in connection with FIGS. 7B and 7C.

The photovoltaic material 712 can be configured to absorb light of any suitable wavelength, such as a wavelength within a range from 50 nm to 800 nm, 50 nm to 300 nm, 50 nm to 500 nm, 100 nm to 200 nm, 100 nm to 300 nm, 100 nm to 400 nm, 100 nm to 500 nm, 100 nm to 600 nm, 200 nm to 400 nm, 200 nm to 500 nm, 300 nm to 700 nm, 300 nm to 800 nm, 400 nm to 700 nm, 400 nm to 720 nm, 400 nm to 800 nm, 500 nm to 800 nm, 700 nm to 800 nm, etc. In some embodiments, the peak absorption wavelength of the photovoltaic material 712 is substantially similar to and/or overlaps the peak emission wavelength of the light produced by the light source. In some embodiments, the photovoltaic material 712 is configured to receive and absorb light within multiple wavelength ranges. For instance, the photovoltaic material 712 can be tuned to at least one, two, three, four, five, or more wavelength ranges. Alternatively or in combination, the energy harvesting mechanism 702a may include a combination of photovoltaic materials 712. For instance, a plurality of different photovoltaic materials 712 may be combined in separate layers, where each layer has a different bandgap such that each layer absorbs light having a different wavelength range (e.g., multijunction photovoltaics).

When the implantable medical device 700a is implanted in the patient's body, the energy harvesting mechanism 702a can generate electrical energy from light received from light sources within the patient's body, e.g., via the photovoltaic effect produced by the photovoltaic material 712. In some embodiments, the energy harvesting mechanism 702a receives light from a naturally-occurring light source within the patient's body, such as native tissues and/or cells of the patient (e.g., tissues and/or cells that have not been genetically modified or otherwise altered from their native state). The native tissues and/or cells may be located proximately to the implantable medical device 700a. For instance, the native tissues and/or cells may be in direct contact with, adjacent to, or otherwise proximate to the implantable medical device 700a (e.g., less than 1 mm, 5 mm, 10 mm, 20 mm, 50 mm, or 100 mm from the implantable medical device 700a).

Native cells may emit biophotons having wavelength within a range from 300 nm to 800 nm, such as 400 nm to 720 nm, as a result of natural cellular processes such as metabolic processes (e.g., cellular respiration). Biophotons may be produced due to the bioluminescent radical and nonradical reactions of Reactive Oxygen Species (ROS) and Reactive Nitrogen Species (RNS), and can involve simple cessation of excited states. Examples include mitochondrial respiration chain and peroxisomal reactions, non-enzymatic and enzymatic lipid peroxidation, oxidation of catecholamines, and oxidation of tyrosine and tryptophan residues in proteins. In some embodiments, the light is produced by one or more of the following native cell types: myocytes (e.g., cardiomyocytes, skeletal myocytes, smooth myocytes), epithelial cells, endothelial cells (e.g., cardiac endothelial cells, vascular endothelial cells), epidermal cells, fibroblasts, adipocytes, osteoblasts, osteoclasts, chondrocytes, erythrocytes, lymphocytes (e.g., T cells, B cells, natural killer cells), hepatocytes, neurons, glial cells, interstitial cells, stem cells, etc. The native cell types may be cell types located at the implantation site of the implantable medical device 700a (e.g., subcutaneous tissue cell types, cardiac tissue cell types).

Alternatively or in combination, the biophoton-emitting tissues may be the result of alteration of tissues and/or cells within the patient, such as via genetic modification of cells to cause the cells to express a light-emitting compound (e.g., a fluorescent or luminescent protein). The light-emitting compound can be a molecule that emits light having a wavelength within a range from 50 nm to 800 nm, 50 nm to 300 nm, 50 nm to 500 nm, 100 nm to 200 nm, 100 nm to 300 nm, 100 nm to 400 nm, 100 nm to 500 nm, 100 nm to 600 nm, 200 nm to 400 nm, 200 nm to 500 nm, 300 nm to 700 nm, 300 nm to 800 nm, 400 nm to 700 nm, 400 nm to 720 nm, 400 nm to 800 nm, 500 nm to 800 nm, 700 nm to 800 nm, etc. Examples of fluorescent proteins include blue fluorescent proteins (e.g., Sirius, Azurite, EBFP, EBFP2, mTagBFP), cyan fluorescent proteins (e.g., ECFP, Cerulean, CyPet, SCFP, TagCFP, AmCyan, Midoriishi Cyan, mTFP1), green fluorescent proteins (e.g., EGFP, Emerald, Superfolder avGFP, T-Sapphire, Azami Green, mWasabi, ZsGreen, TagGFP, TagGFP2, TurboGFP, CopGFP, AceGFP), yellow fluorescent proteins (e.g., EYFP, Topaz, Venus, Citrine, YPet, SYFP, mAmetrine, TagYFP, TurboYFP, ZsYellow, PhiYFP), orange fluorescent proteins (e.g., Kusabira Orange, Kusabira Orange2, mOrange, mOrange2, dTomato, dTomato-Tandem, DsRed, DsRed2, DsRed-Express (T1), DsRed-Express2, DsRed-Max, DsRed-Monomer, TurboRFP, TagRFP, TagRFP-T), red fluorescent proteins (e.g., mRuby, mApple, mStrawberry, AsRed2, mRFP1, jRed, mCherry, eqFP611, tdRFP611, HcRed1, mRaspberry), and far-red fluorescent proteins (e.g., tdRFP639, mKate, mKate2, Katushka, tdKatushka, HcRed-Tandem, mPlum, AQ6143). Examples of luminescent proteins include luciferases, such as luciferases of Photinus pyralis, Luciola cruciate, Luciola italic, Luciola lateralis, Luciola mingrelica, Photuris pennsylvanica, Pyrophorus plagiophthalamus, Phrixothrix hirtus, Renilla reniformis, Gaussia princeps, Cypridina noctiluca, Cypridina hilgendorfii, Metridia longa, and Oplophorus gracilorostris.

Any of the native cell types described herein may be genetically modified to express a light-emitting compound. Genetic modification of cells may be performed using any technique known to those of skill, including viral transfection and non-viral transfection techniques, and/or transient and stable transfection techniques. In some embodiments, cells are transfected with a polynucleotide (e.g., DNA or RNA) encoding the light-emitting compound. The polynucleotide can be naked or can be delivered via a viral vector (e.g., a lentiviral vector, an adeno-associated viral vector, a vaccinia viral vector, a poxvirus viral vector, a herpes viral vector, an alphavirus viral vector, gamma retrovirus, a polyoma viral vector) or a nonviral vector (e.g., a polymer, a polymer nanoparticle, a liposome, a lipid, a lipid nanoparticle). The polynucleotide may be introduced into a cell using physical or chemical methods. In some embodiments, a polynucleotide is introduced into a cell using calcium phosphate precipitation, lipofection, particle bombardment, microinjection, electroporation, and the like. In some embodiments, the polynucleotide expression cassette may be introduced using colloidal dispersion systems (e.g., macromolecule complexes), nanocapsules, microspheres, beads, lipid-based systems (e.g., oil-in-water emulsions, micelles, mixed micelles, liposomes), and the like. Genetic modification may be performed before, concurrently with, or after implantation of the implantable medical device 700a in the patient's body. In some embodiments, the genetic modification is performed only on tissues and/or cells that are in direct contact with, adjacent to, or otherwise in close proximity to the implantable medical device 700a. In some embodiments, the genetic modification is performed only on tissue and/or cells that are in direct contact with, adjacent to, or otherwise in close proximity to the photovoltaic material 712.

The electrical energy produced by the energy harvesting mechanism 702a can be delivered to one or more of the device components 710 via the electrical circuitry 708 to power the operation of the device components 710. In some embodiments, the electrical circuitry 708 includes filters, amplifiers, resistors, capacitors, inductors, transistors, and/or other circuit elements for regulating the electrical energy passed to the device components 710. In some embodiments, the one or more device components 710 includes a power source (e.g., the power source 252 of the implantable medical device 200 of FIG. 2B or the power source 620 of the device 600 of FIG. 6), and the electrical energy is used to recharge the power source of the implantable medical device 700. Alternatively, the electrical energy may directly power the one or more device components 710. In some embodiments, the device component 710 can be related to device functions for monitoring and/or stimulating the patient's heart. For instance, the device component 710 can be or include electrodes configured to sense electrical signals from the patient, deliver electrical stimulation to the patient, or a combination thereof. The electrical circuitry 708 may include corresponding circuitry for instructing the electrodes, such as the sensing circuitry 608 and/or the therapy generation circuitry 610 of the device 600 of FIG. 6. Alternatively or in combination, the device component 710 can be related to device functions for communicating to external devices, such as the communication circuitry 616 of the device 600 of FIG. 6. Alternatively or in combination, the device component 710 can be related to power management, such as power management circuitry.

The components of the implantable medical device 700a illustrated in FIG. 7A can be modified in many different ways. For example, any of the components shown in FIG. 7A can be combined with each other, e.g., the one or more device components 710 can be incorporated into the electrical circuitry 708. Any of the components shown in FIG. 7A can be divided into smaller subcomponents. The implantable medical device 700a can also include additional components not shown in FIG. 7A. For example, the implantable medical device 700a can include power management or other circuitry configured to monitor an energy status (e.g., charge level, charging rate, net power into and/or out of a power source, remaining battery life, conversion efficiency, device component utilization, etc.).

Moreover, as noted above, although FIG. 7A illustrates the energy harvesting mechanism 702a as covering the entire housing 704, other configurations are possible, e.g., the energy harvesting mechanism 702a may be disposed on only a portion of the housing 704, may be located within the housing 704, etc.

For instance, FIG. 7B is a conceptual cross-sectional diagram of an implantable medical device 700b including an energy harvesting mechanism 702b, in accordance with embodiments of the present technology. The implantable medical device 700b is identical to the implantable medical device 700a of FIG. 7A, except that the photovoltaic material 712 of the energy harvesting mechanism 702b is disposed on only a portion of the housing 704. For example, as shown in FIG. 7B, the photovoltaic material 712 may be disposed on a first portion 714 of the upper surface 706a, whereas the photovoltaic material 712 may not be disposed on a second portion 716 of the upper surface 706a. In some embodiments, the first portion 714 corresponds to a structural feature that has a high likelihood of receiving light. For instance, when the implantable medical device 700b is positioned within the patient's body, the first portion 714 may be positioned proximately to and/or in direct contact with a light source within the patient's body (e.g., a tissue and/or cell that naturally emits light or that has been modified to emit light). Alternatively or in combination, the first portion 714 may correspond to a non-conductive surface of the implanted medical device 700b, such as a sapphire-coated surface, whereas the second portion 716 may not correspond to a non-conductive surface of the implanted medical device 700b. Alternatively or in combination, the first portion 714 may avoid functional regions of the implantable medical device 700b, such as electrodes, sensors, indicators, antenna, etc.

Alternatively or in combination, the photovoltaic material 712 may be located on other portions of the housing 704, such as on the entire upper surface 706a, on the entire lower surface 706b, on only a portion of the lower surface 706b, on all of the side surfaces 706c, on only a portion of the side surfaces 706c, or suitable combinations thereof. The location of the photovoltaic material 712 may be selected based on the configuration of the implantable medical device 700b, the position and/or orientation of the implantable medical device 700b, the types of tissues and/or cells present at the implantation site, etc.

The embodiments of FIGS. 7A and 7B may be advantageous in situations where the implantable medical device 700a, 700b is implanted mostly or entirely within solid tissue that serves as the light source (e.g., subcutaneous implantation), such that the photovoltaic material 712 is in direct contact with or in close proximity to the solid tissue. However, the embodiments of FIGS. 7A and 7B may also be used for other applications and/or implantation sites.

FIG. 7C is a conceptual cross-sectional diagram of an implantable medical device 700c including an energy harvesting mechanism 702c, in accordance with embodiments of the present technology. The implantable medical device 700c is identical to the implantable medical device 700a of FIG. 7A, except that the photovoltaic material 712 of the energy harvesting mechanism 702c is disposed on one or more additional components of the housing 704 of the implantable medical device 700c that are configured to be in direct contact with (e.g., embedded in) a tissue that emits light. In the illustrated embodiment, the photovoltaic material 712 is disposed on one or more fixation mechanisms 718 (e.g., the same or similar to the fixation mechanism 418 of the pacing device 400 of FIG. 4) extending from the housing 704. The fixation mechanism 718 may be configured to anchor the implantable medical device 700c to tissue of the patient. In some embodiments, the fixation mechanism 718 may include one or more of tines, barbs, coils (e.g., helices), darts, or hooks. In some embodiments, the photovoltaic material 712 is disposed over the entirety of the fixation mechanism 718. Alternatively, the photovoltaic material 712 can be disposed over less than the entirety of the fixation mechanism 718. For instance, the photovoltaic material 712 may be disposed only on portions of the fixation mechanism 718 that are configured to contact and/or face the patient's surrounding tissue. In embodiments where the fixation mechanism 718 includes functional portions (e.g., an electrode), the photovoltaic material 712 may be disposed away from the functional portions. Further, the photovoltaic material 712 may be located on an additional component that is not the fixation mechanism 718, such as on another component that serves a different functional purpose or exists solely to provide a support for the photovoltaic material 712. For instance, the photovoltaic material 712 may be disposed on a protrusion or other component extending from the housing 704.

The embodiment of FIG. 7C may be advantageous in situations where the implantable medical device 700c is implanted intravascularly or in a heart chamber, such that the housing 704 of the implantable medical device is surrounded by blood or other physiological fluids which may attenuate light, and the fixation mechanism 718 or other component is in direct contact with a solid tissue that serves as the light source (e.g., cardiac tissue). However, the embodiment of FIG. 7C may also be used for other applications and/or implantation sites.

Although FIGS. 7A-7C illustrate energy harvesting mechanisms including photovoltaic materials that are located on an external portion of an implantable medical device, this is not intended to be limiting. An energy harvesting mechanism can alternatively or additionally be located in an internal portion of an implantable medical device, such as within the housing of the device. In such embodiments, the housing may include an optically transparent or translucent substrate (e.g., a window) that allows light from a light source to enter the housing. The photovoltaic material can be positioned proximate to (e.g., in direct contact with) the window to receive and absorb the light.

In some embodiments, the light source for the implantable medical device is a non-naturally occurring light source including one or more light-emitting species that have been introduced into the patient's body. The light-emitting species can be a cell or microorganism (e.g., bacteria, fungi) that natively emits light or that has been genetically modified to emit light. In some embodiments, the light-emitting species is an immortalized mammalian cell line derived from any of the following cell types: myocytes (e.g., cardiomyocytes, skeletal myocytes, smooth myocytes), epithelial cells, endothelial cells (e.g., cardiac endothelial cells, vascular endothelial cells), epidermal cells, fibroblasts, adipocytes, osteoblasts, osteoclasts, chondrocytes, erythrocytes, lymphocytes (e.g., T cells, B cells, natural killer cells), hepatocytes, neurons, glial cells, interstitial cells, stem cells, etc. In some embodiments, the light-emitting species is a non-pathogenic microorganism derived from Bacillus coagulans, Bifidobacterium adolescentis, Bifidobacterium animalis, Bifidobacterium bifidum, Bifidobacterium breve, Bifidobacterium essencis, Bifidobacterium faecium, Bifidobacterium infantis, Bifidobacterium lactis, Bifidobacterium longum, Bifidobacterium longum subsp. infantis, Bifidobacterium pseudolungum, Lactobacillus acidophilus, Lactobacillus boulardii, Lactobacillus breve, Lactobacillus brevis, Lactobacillus bulgaricus, Lactobacillus casei, Lactobacillus delbrueckii ssp. Bulgaricus, Lactobacillus fermentum, Lactobacillus gasseri, Lactobacillus helveticus, Lactobacillus paracasei, Lactobacillus plantarum, Lactobacillus reuteri, Lactobacillus rhamnosus, Lactobacillus rhamnosus GG, Lactobacillus salivarius, Lactococcus lactis, Streptococcus thermophilus, Pediococcus acidilactici, Enterococcus faecium, Leuconostoc, Carnobacterium, Proprionibacterium, Saccharomyces boulardii, or Escherichia coli.

In embodiments where a light-emitting species is used as the light source, the light-emitting species can be contained in a bioreactor to allow for long-term proliferation and survival of the light-emitting species while also preventing the species from coming into contact with the native tissues of the patient's body (e.g., for safety reasons). The bioreactor can be implanted in the patient's body, e.g., as part of an implantable medical device including an energy harvesting mechanism.

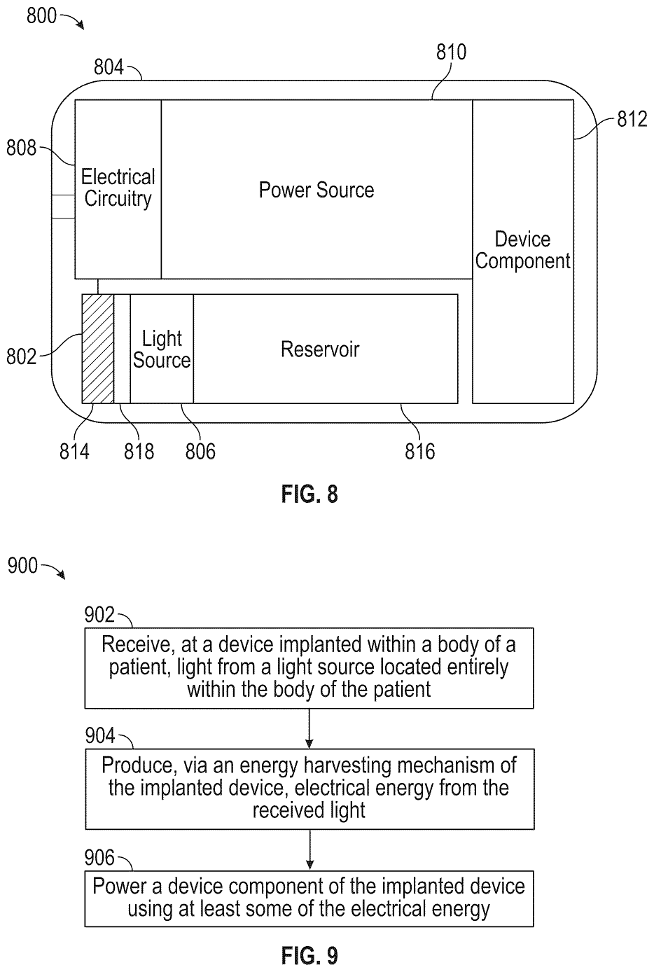

FIG. 8 is a conceptual cross-sectional diagram of an implantable medical device 800 including an energy harvesting mechanism 802, in accordance with embodiments of the present technology. The implantable medical device 800 is an example of a medical device 102 that may be used in the system 100 of FIG. 1. The implantable medical device 800 can be an insertable cardiac monitor for monitoring activity of a patient's heart (e.g., as described in connection with FIGS. 2A-3) and/or a pacing device configured to provide electrical stimulation to the patient's heart (e.g., as described in connection with FIGS. 4-6). Accordingly, the implantable medical device 800 may additionally or alternatively include any of the components of the devices described herein, such as any of the devices described in connection with FIGS. 1-6. Moreover, the implantable medical device 800 need not be used for monitoring and/or stimulating the heart, and can be used for other therapeutic applications and/or elsewhere in the body.

In some embodiments, the implantable medical device 800 includes a housing 804 defining an interior cavity containing the energy harvesting mechanism 802, a light source 806, and other components of the implantable medical device 800, such as electrical circuitry 808, a power source 810, and additional device components 812. The energy harvesting mechanism 802 can produce electrical energy that is delivered to the power source 810 and/or additional device components 812 via the electrical circuitry 808.

In some embodiments, the energy harvesting mechanism 802 includes one or more photovoltaic cells 814 that convert light energy into electrical energy. Similarly to the photovoltaic material 712 of the implantable medical devices 700a-700c of FIGS. 7A-7C, the photovoltaic cell 814 can include any of a plurality of photovoltaic materials configured to produce the photovoltaic effect. In some embodiments, the photovoltaic material includes a semiconductor such as silicon. The silicon can be a monocrystalline silicon or a polycrystalline silicon, or a combination thereof. Alternatively or in combination, the photovoltaic material can include other semiconductors, such as gallium arsenide, cadmium telluride (CdTe), copper indium gallium selenide (CIGS), etc. Other photovoltaic materials are possible, such as perovskite photovoltaics (e.g., methylammonium lead triiodide (MAPbI₃), organic photovoltaics, etc. Further, additional optical materials may be used to enhance the absorption of the photovoltaic material. For instance, a mirror or lens may be used to concentrate and/or direct light toward the photovoltaic material.

The photovoltaic cell 814 can have any suitable geometry. For instance, the photovoltaic cell 814 may have a flat, elongate body. Alternatively, the photovoltaic cell 814 may be cylindrical or any other suitable shape. In some embodiments, the photovoltaic cell 814 includes a thin film of photovoltaic materials. Alternatively or in combination, the photovoltaic cell 814 may include a plurality of layers, where each layer is tuned to receive a different wavelength of light. Further, the photovoltaic cell 814 may include additional or alternative components such as electrical contacts and/or substrates. In some embodiments, the photovoltaic cell 814 may be a first photovoltaic cell, and the implantable medical device 800 may include additional photovoltaic cells.

The photovoltaic cell 814 can be configured to absorb and/or process light of any suitable wavelength, such as a wavelength within a range from 50 nm to 800 nm, 50 nm to 300 nm, 50 nm to 500 nm, 100 nm to 200 nm, 100 nm to 300 nm, 100 nm to 400 nm, 100 nm to 500 nm, 100 nm to 600 nm, 200 nm to 400 nm, 200 nm to 500 nm, 300 nm to 700 nm, 300 nm to 800 nm, 400 nm to 700 nm, 400 nm to 720 nm, 400 nm to 800 nm, 500 nm to 800 nm, 700 nm to 800 nm, etc. In some embodiments, the peak absorption wavelength of a photovoltaic material within the photovoltaic cell 814 is substantially similar to and/or overlaps the peak emission wavelength of the light produced by the light source. In some embodiments, the photovoltaic cell 814 is configured to receive and absorb light within multiple wavelength ranges. For instance, the photovoltaic cell 814 may have a photovoltaic material tuned to at least one, two, three, four, five, or more wavelength ranges. Alternatively or in combination, the photovoltaic cell 814 may have a plurality of photovoltaic layers, where each layer has a different bandgap such that each layer absorbs light having a different wavelength range (e.g., multijunction photovoltaics).

When the implantable medical device 800 is implanted in the patient's body, the energy harvesting mechanism 802 can generate electrical energy from the light source 806 within the implantable medical device 800. The light source 806 can be a bioreactor (e.g., a hermetically-sealed environment) that includes a light-emitting species. The light-emitting species can be any of the embodiments described herein, such as cells or microorganisms that naturally emit light or have been genetically engineered to emit light, e.g., via expression of a light-emitting compound such as a fluorescent or luminescent protein as described elsewhere herein. The light-emitting compound can be a molecule that emits light having a wavelength within a range from 50 nm to 800 nm, 50 nm to 300 nm, 50 nm to 500 nm, 100 nm to 200 nm, 100 nm to 300 nm, 100 nm to 400 nm, 100 nm to 500 nm, 100 nm to 600 nm, 200 nm to 400 nm, 200 nm to 500 nm, 300 nm to 700 nm, 300 nm to 800 nm, 400 nm to 700 nm, 400 nm to 720 nm, 400 nm to 800 nm, 500 nm to 800 nm, 700 nm to 800 nm, etc. Examples of fluorescent proteins include blue fluorescent proteins (e.g., Sirius, Azurite, EBFP, EBFP2, mTagBFP), cyan fluorescent proteins (e.g., ECFP, Cerulean, CyPet, SCFP, TagCFP, AmCyan, Midoriishi Cyan, mTFP1), green fluorescent proteins (e.g., EGFP, Emerald, Superfolder avGFP, T-Sapphire, Azami Green, mWasabi, ZsGreen, TagGFP, TagGFP2, TurboGFP, CopGFP, AceGFP), yellow fluorescent proteins (e.g., EYFP, Topaz, Venus, Citrine, YPet, SYFP, mAmetrine, TagYFP, TurboYFP, ZsYellow, PhiYFP), orange fluorescent proteins (e.g., Kusabira Orange, Kusabira Orange2, mOrange, mOrange2, dTomato, dTomato-Tandem, DsRed, DsRed2, DsRed-Express (T1), DsRed-Express2, DsRed-Max, DsRed-Monomer, TurboRFP, TagRFP, TagRFP-T), red fluorescent proteins (e.g., mRuby, mApple, mStrawberry, AsRed2, mRFP1, jRed, mCherry, eqFP611, tdRFP611, HcRed1, mRaspberry), and far-red fluorescent proteins (e.g., tdRFP639, mKate, mKate2, Katushka, tdKatushka, HcRed-Tandem, mPlum, AQ143). Examples of luminescent proteins include luciferases, such as luciferases of Photinus pyralis, Luciola cruciate, Luciola italic, Luciola lateralis, Luciola mingrelica, Photuris pennsylvanica, Pyrophorus plagiophthalamus, Phrixothrix hirtus, Renilla reniformis, Gaussia princeps, Cypridina noctiluca, Cypridina hilgendorfii, Metridia longa, and Oplophorus gracilorostris.

In some embodiments, the light-emitting species is an immortalized mammalian cell line derived from any of the following cell types: myocytes (e.g., cardiomyocytes, skeletal myocytes, smooth myocytes), epithelial cells, endothelial cells (e.g., cardiac endothelial cells, vascular endothelial cells), epidermal cells, fibroblasts, adipocytes, osteoblasts, osteoclasts, chondrocytes, erythrocytes, lymphocytes (e.g., T cells, B cells, natural killer cells), hepatocytes, neurons, glial cells, interstitial cells, stem cells, etc. In some embodiments, the light-emitting species is a non-pathogenic microorganism derived from Bacillus coagulans, Bifidobacterium adolescentis, Bifidobacterium animalis, Bifidobacterium bifidum, Bifidobacterium breve, Bifidobacterium essencis, Bifidobacterium faecium, Bifidobacterium infantis, Bifidobacterium lactis, Bifidobacterium longum, Bifidobacterium longum subsp. infantis, Bifidobacterium pseudolungum, Lactobacillus acidophilus, Lactobacillus boulardii, Lactobacillus breve, Lactobacillus brevis, Lactobacillus bulgaricus, Lactobacillus casei, Lactobacillus delbrueckii ssp. Bulgaricus, Lactobacillus fermentum, Lactobacillus gasseri, Lactobacillus helveticus, Lactobacillus paracasei, Lactobacillus plantarum, Lactobacillus reuteri, Lactobacillus rhamnosus, Lactobacillus rhamnosus GG, Lactobacillus salivarius, Lactococcus lactis, Streptococcus thermophilus, Pediococcus acidilactici, Enterococcus faecium, Leuconostoc, Carnobacterium, Proprionibacterium, Saccharomyces boulardii, or Escherichia coli.

In some embodiments, the light-emitting species is self-sustaining. Stated differently, the light-emitting species may survive in the bioreactor for long periods of time, such as for a substantial portion or the entirety of the lifetime of the implantable medical device 800. In some embodiments, the bioreactor includes or is coupled to a reservoir 816. The reservoir 816 may include reagents (e.g., culture media) configured to supply nutrients to the light-emitting species to support cell proliferation over extended periods of time. Further, the bioreactor may be configured to provide suitable conditions for the light-emitting species, such as by maintaining an appropriate temperature, pH, oxygen level, etc.

The light produced by the light-emitting species of the light source 806 can be absorbed by the photovoltaic cell 814 of the energy harvesting mechanism 802, thereby resulting in production of electrical energy by the photovoltaic cell 814. Optionally, an optically transparent or translucent substrate 818 (e.g., a window) may be positioned between the photovoltaic cell 814 and the light source 806.

The electrical energy produced by the energy harvesting mechanism 802 can be delivered to the power source 810 and/or the other device components 812 via the electrical circuitry 808 to power the operation thereof. In some embodiments, the electrical circuitry 808 includes a charge controller that is configured to regulate the electrical energy such that the electrical energy is suitable for charging the power source 810 and/or directly powering the other device components 812. The charge controller 820 may include filters, amplifiers, resistors, capacitors, inductors, transistors, and/or other circuit elements for regulating the electrical energy passed to the power source 810 and/or other device components 812.

In some embodiments, the electrical energy is used to recharge the power source 810. Alternatively, the electrical energy may directly power the one or more other device components 812. In some embodiments, the other device components 812 can be related to device functions for monitoring and/or stimulating the patient's heart. For instance, the other device components 812 can be or include electrodes configured to sense electrical signals from the patient, deliver electrical stimulation to the patient, or a combination thereof. The other device components 812 may include corresponding circuitry for instructing the electrodes, such as the sensing circuitry 608 and/or the therapy generation circuitry 610 of the device 600 of FIG. 6. Alternatively or in combination, the other device components 812 can be related to device functions for communicating to external devices, such as the communication circuitry 616 of the device 600 of FIG. 6. Alternatively or in combination, the other device components 812 can be related to power management, such as power management circuitry.

The components of the implantable medical device 800 illustrated in FIG. 8 can be modified in many different ways. For example, any of the components shown in FIG. 8 can be combined with each other, e.g., the power source 810 can be incorporated into the electrical circuitry 808. Any of the components shown in FIG. 8 can be divided into smaller subcomponents. Some of the components in FIG. 8 are optional and may be omitted (e.g., the power source 810). The implantable medical device 800 can also include additional components not shown in FIG. 8. For example, the electrical circuitry 808 can include power management circuitry. In some embodiments, an energy status (e.g., charge level, charging rate, net power into and/or out of the power source, remaining battery life, conversion efficiency, device component utilization, etc.) of the implantable medical device 800 may be provided by the electrical circuitry 808 or other component.

FIG. 9 is a flow diagram illustrating a method 900 for energy harvesting in an implantable medical device, in accordance with embodiments of the present technology. The method 900 can be performed using any of the systems and devices described herein, such as any of the devices of FIGS. 1-8. In some embodiments, some or all of the processes of the method 900 are implemented as computer-readable instructions (e.g., program code) that are configured to be executed by one or more processors (e.g., processing circuitry 614 of the device 600 of FIG. 6).

The method 900 can begin at block 902 with receiving, at a medical device implanted within a body of a patient, light from a light source located entirely within the body of the patient. The medical device can be any of the devices described herein (e.g., the devices of FIGS. 1-8). In some embodiments, the implanted medical device is an insertable cardiac monitor (e.g., as described above in connection with FIGS. 2A-3) and/or a leadless pacemaker (e.g., as described above in connection with FIGS. 4-6). Other medical devices are possible, such as a medical device used elsewhere in the body.

The light can be received from a light source within the patient's body. The light source may be a naturally-occurring light source that is external to the medical device, such as native tissues and/or cells of the patient (e.g., tissues and/or cells that have not been genetically modified or otherwise altered from their native state). For instance, the native tissues and/or cells may be located proximately to the implantable medical device, and the native tissues and/or cells may emit biophotons having wavelength within a range from 300 nm to 800 nm, such as 400 nm to 720 nm, e.g., as a result of natural cellular processes such as metabolic processes (e.g., cellular respiration). In some embodiments, the light is produced by one or more of the following native cell types: myocytes (e.g., cardiomyocytes, skeletal myocytes, smooth myocytes), epithelial cells, endothelial cells (e.g., cardiac endothelial cells, vascular endothelial cells), epidermal cells, fibroblasts, adipocytes, osteoblasts, osteoclasts, chondrocytes, erythrocytes, lymphocytes (e.g., T cells, B cells, natural killer cells), hepatocytes, neurons, glial cells, interstitial cells, stem cells, etc.

Alternatively or in combination, the light source may be a non-naturally-occurring light source that is external to the medical device, such as non-native tissues and/or cells within the patient that have been altered via genetic modification to express a light-emitting compound (e.g., a fluorescent or luminescent protein). The light-emitting compound can be a molecule that emits light having a wavelength within a range from 50 nm to 800 nm, 50 nm to 300 nm, 50 nm to 500 nm, 100 nm to 200 nm, 100 nm to 300 nm, 100 nm to 400 nm, 100 nm to 500 nm, 100 nm to 600 nm, 200 nm to 400 nm, 200 nm to 500 nm, 300 nm to 700 nm, 300 nm to 800 nm, 400 nm to 700 nm, 400 nm to 720 nm, 400 nm to 800 nm, 500 nm to 800 nm, 700 nm to 800 nm, etc. Examples of fluorescent proteins include blue fluorescent proteins (e.g., Sirius, Azurite, EBFP, EBFP2, mTagBFP, etc.) cyan fluorescent proteins (e.g., ECFP, Cerulean, CyPet, SCFP, TagCFP, AmCyan, Midoriishi Cyan, mTFP1 etc.), green fluorescent proteins (e.g., EGFP, Emerald, Superfolder avGFP, T-Sapphire, Azami Green, mWasabi, ZsGreen, TagGFP, TagGFP2, TurboGFP, CopGFP, AceGFP, etc.), yellow fluorescent proteins (e.g., EYFP, Topaz, Venus, Citrine, YPet, SYFP, mAmetrine, TagYFP, TurboYFP, ZsYellow, PhiYFP, etc.), orange fluorescent proteins (e.g., Kusabira Orange, Kusabira Orange2, mOrange, mOrange2, dTomato, dTomato-Tandem, DsRed, DsRed2, DsRed-Express (T1), DsRed-Express2, DsRed-Max, DsRed-Monomer, TurboRFP, TagRFP, TagRFP-T, etc.), red fluorescent proteins (e.g., mRuby, mApple, mStrawberry, AsRed2, mRFP1, jRed, mCherry, eqFP611, tdRFP611, HcRed1, mRaspberry, etc.), and far-red fluorescent proteins (e.g., tdRFP639, mKate, mKate2, Katushka, tdKatushka, HcRed-Tandem, mPlum, AQ143, etc.). Examples of luminescent proteins include luciferases, such as Photinus pyralis, Luciola cruciate, Luciola italic, Luciola lateralis, Luciola mingrelica, Photuris pennsylvanica, Pyrophorus plagiophthalamus, Phrixothrix hirtus, Renilla reniformis, Gaussia princeps, Cypridina noctiluca, Cypridina hilgendorfii, Metridia longa, and Oplophorus gracilorostris.

Any of the native cell types described herein may be genetically modified to express a light-emitting compound. Genetic modification of cells may be performed using any technique known to those of skill, including viral transfection and non-viral transfection techniques, and/or transient and stable transfection techniques.

Alternatively or in combination, the light source may be received from a light source that is within the implanted medical device. In some embodiments, the implanted medical device may include a bioreactor, e.g., as described above in connection with FIG. 8. For instance, the bioreactor can be a hermetically-sealed environment that includes a light-emitting species. The light-emitting species can be any of the embodiments described herein, such as cells or microorganisms that naturally emit light or have been genetically engineered to emit light, e.g., via expression of a light-emitting compound such as a fluorescent or luminescent protein as described elsewhere herein. The light-emitting species can be an immortalized mammalian cell line derived from any of the following cell types: myocytes (e.g., cardiomyocytes, skeletal myocytes, smooth myocytes), epithelial cells, endothelial cells (e.g., cardiac endothelial cells, vascular endothelial cells), epidermal cells, fibroblasts, adipocytes, osteoblasts, osteoclasts, chondrocytes, erythrocytes, lymphocytes (e.g., T cells, B cells, natural killer cells), hepatocytes, neurons, glial cells, interstitial cells, stem cells, etc. In some embodiments, the light-emitting species is a non-pathogenic microorganism derived from Bacillus coagulans, Bifidobacterium adolescentis, Bifidobacterium animalis, Bifidobacterium bifidum, Bifidobacterium breve, Bifidobacterium essencis, Bifidobacterium faecium, Bifidobacterium infantis, Bifidobacterium lactis, Bifidobacterium longum, Bifidobacterium longum subsp. infantis, Bifidobacterium pseudolungum, Lactobacillus acidophilus, Lactobacillus boulardii, Lactobacillus breve, Lactobacillus brevis, Lactobacillus bulgaricus, Lactobacillus casei, Lactobacillus delbrueckii ssp. Bulgaricus, Lactobacillus fermentum, Lactobacillus gasseri, Lactobacillus helveticus, Lactobacillus paracasei, Lactobacillus plantarum, Lactobacillus reuteri, Lactobacillus rhamnosus, Lactobacillus rhamnosus GG, Lactobacillus salivarius, Lactococcus lactis, Streptococcus thermophilus, Pediococcus acidilactici, Enterococcus faecium, Leuconostoc, Carnobacterium, Proprionibacterium, Saccharomyces boulardii, or Escherichia coli. The light-emitting species can be any species that emits light having a wavelength within a range from 50 nm to 800 nm, 50 nm to 300 nm, 50 nm to 500 nm, 100 nm to 200 nm, 100 nm to 300 nm, 100 nm to 400 nm, 100 nm to 500 nm, 100 nm to 600 nm, 200 nm to 400 nm, 200 nm to 500 nm, 300 nm to 700 nm, 300 nm to 800 nm, 400 nm to 700 nm, 400 nm to 720 nm, 400 nm to 800 nm, 500 nm to 800 nm, 700 nm to 800 nm, etc.

The method 900 can continue at block 904 with producing, via an energy harvesting mechanism of the implanted medical device, electrical energy from the received light. In some embodiments, the energy harvesting mechanism is or includes a photovoltaic material. The photovoltaic material may be disposed on an external surface of the implanted medical device, or may be disposed within the implanted medical device, e.g., depending on the location of the light source. For instance, the photovoltaic material may coat the entirety of the implanted medical device, or less than the entirety of the implanted medical device, e.g., as described above in connection with FIGS. 7A and 7B. Alternatively or in combination, the photovoltaic material may be located on a fixation mechanism or other extending from a housing of the implanted medical device, e.g., as described above in connection with FIG. 7C. Moreover, the photovoltaic material may be disposed within a housing the implanted medical device, e.g., as described above in connection with FIG. 8.

The photovoltaic material can include any of a plurality of materials configured to produce the photovoltaic effect. In some embodiments, the photovoltaic material includes a semiconductor such as silicon. The silicon can be a monocrystalline silicon or a polycrystalline silicon, or a combination thereof. Alternatively or in combination, the photovoltaic material can include other semiconductors, such as gallium arsenide, cadmium telluride (CdTe), copper indium gallium selenide (CIGS), etc. Other photovoltaic materials are possible, such as in perovskite photovoltaics (e.g., methylammonium lead triiodide (MAPbI₃), organic photovoltaics, etc. Further, additional optical materials may be used to enhance the absorption of the photovoltaic material. For instance, a mirror or lens may be used to concentrate and/or direct light toward the photovoltaic material.

The photovoltaic material can be configured to absorb light of any suitable wavelength, such as within a range from 50 nm to 800 nm, 50 nm to 300 nm, 50 nm to 500 nm, 100 nm to 200 nm, 100 nm to 300 nm, 100 nm to 400 nm, 100 nm to 500 nm, 100 nm to 600 nm, 200 nm to 400 nm, 200 nm to 500 nm, 300 nm to 700 nm, 300 nm to 800 nm, 400 nm to 700 nm, 400 nm to 720 nm, 400 nm to 800 nm, 500 nm to 800 nm, 700 nm to 800 nm, etc. In some embodiments, the peak absorption wavelength of the photovoltaic material is substantially similar to and/or overlaps the peak emission wavelength of the light produced by the light source. In some embodiments, the photovoltaic material is configured to receive and absorb light within multiple wavelength ranges. For instance, the photovoltaic material can be tuned to at least one, two, three, four, five, or more wavelength ranges. Alternatively or in combination, the energy harvesting mechanism may include a combination of photovoltaic materials. For instance, a plurality of different photovoltaic materials may be combined in separate layers, where each layer has a different bandgap such that each layer absorbs light having a different wavelength range (e.g., multijunction photovoltaics).

The method 900 can continue at block 906 with powering a device component of the implanted medical device using at least some of the electrical energy. In some embodiments, the device component includes a power source (e.g., the power source 252 of the implantable medical device 200 of FIG. 2B or the power source 620 of the device 600 of FIG. 6). Optionally, the power source may be rechargeable, and the at least some of the electrical energy may be used to recharge the power source. Alternatively or in combination, the device component can be related to device functions for monitoring and/or stimulating the patient's heart. For instance, the device component can be or include electrodes configured to sense electrical signals from the patient, deliver electrical stimulation to the patient, or a combination thereof. Alternatively or in combination, the device component can be related to device functions for communicating to external devices. Alternatively or in combination, the device component can be related to power management, such as power management circuitry.

### EXAMPLES

The following examples are included to further describe some aspects of the present technology, and should not be used to limit the scope of the technology.

Example 1. An implantable medical device comprising:
a housing configured to be implanted in a body of a patient;
an energy harvesting mechanism carried by the housing, the energy harvesting mechanism configured to receive light from a light source located entirely within the body of the patient and to produce electrical energy from the received light; and
electrical circuitry coupled to the energy harvesting mechanism, wherein the electrical circuitry is configured to deliver at least some of the electrical energy to a device component carried by the housing to power the device component.

Example 2. The implantable medical device of Example 1, wherein the light source comprises a tissue of the body of the patient, and wherein the light comprises biophotons emitted by the tissue.

Example 3. The implantable medical device of Example 2, wherein the biophotons comprise a wavelength within a range from 400 nm to 720 nm.

Example 4. The implantable medical device of Example 2 or 3, wherein the tissue comprises native cells of the patient that emit the biophotons.

Example 5. The implantable medical device of any one of Examples 2 to 4, wherein the tissue comprises cells of the patient that are genetically engineered to emit the biophotons.

Example 6. The implantable medical device of any one of Examples 2 to 5, wherein the light is produced during metabolic processes.

Example 7. The implantable medical device of any one of Examples 1 to 6, wherein the light source comprises a bioreactor disposed within the housing, the bioreactor comprising a light-emitting species.

Example 8. The implantable medical device of any one of Examples 1 to 7, wherein the energy harvesting mechanism comprises a photovoltaic material.

Example 9. The implantable medical device of Example 8, wherein the photovoltaic material is located on an external surface of the housing.

Example 10. The implantable medical device of Example 8 or 9, further comprising a fixation mechanism extending from the housing, the fixation mechanism configured to anchor the implantable medical device to tissue of the patient, wherein the photovoltaic material is located on the fixation mechanism.

Example 11. The implantable medical device of Example 10, wherein the fixation mechanism comprises one or more of tines, barbs, coils, darts, or hooks.

Example 12. The implantable medical device of any one of Examples 8 to 11, wherein the photovoltaic material comprises silicon, gallium arsenide, cadmium telluride (CdTe), copper indium gallium selenide (CIGS), or methylammonium lead triiodide (MAPbI₃).

Example 13. The implantable medical device of any one of Examples 8 to 12, wherein the housing comprises a window, and wherein the photovoltaic material is disposed within the housing proximate to the window.

Example 14. The implantable medical device of any one of Examples 1 to 13, wherein the device component comprises a rechargeable power source.

Example 15. The implantable medical device of any one of Examples 1 to 14, wherein the device component comprises one or more electrodes configured to sense electrical signals from the patient, deliver electrical stimulation to the patient, or a combination thereof.

Example 16. The implantable medical device of any one of Examples 1 to 15, wherein the implantable medical device is an insertable cardiac monitor or a leadless pacemaker.

Example 17. The implantable medical device of any one of Examples 1 to 16, wherein the implantable medical device is configured to be implanted subcutaneously.

Example 18. A method comprising:
receiving, at a medical device implanted within a body of a patient, light from a light source located entirely within a body of a patient;
producing, via an energy harvesting mechanism of the implanted medical device, electrical energy from the received light; and
powering a device component of the implanted medical device using at least some of the electrical energy.

Example 19. The method of Example 18, wherein the light source comprises a tissue of the body of the patient, and wherein the light comprises biophotons emitted by the tissue.

Example 20. The method of Example 19, wherein the biophotons comprise a wavelength within a range from 400 nm to 720 nm.

Example 21. The method of Example 19 or 20, wherein the tissue comprises native cells of the patient that emit the biophotons.

Example 22. The method of any one of Examples 19 to 21, wherein the tissue comprises cells of the patient that are genetically engineered to emit the biophotons.

Example 23. The method of any one of Examples 19 to 22, wherein the light is produced during metabolic processes.

Example 24. The method of any one of Examples 18 to 23, wherein the light source comprises a bioreactor disposed within the implanted medical device, the bioreactor comprising a light-emitting species.

Example 25. The method of any one of Examples 18 to 24, wherein the energy harvesting mechanism comprises a photovoltaic material.

Example 26. The method of Example 25, wherein the photovoltaic material is located on an external surface of the implanted medical device.

Example 27. The method of Example 25 or 26, wherein the implanted medical device comprises a housing and a fixation mechanism extending from the housing, wherein the fixation mechanism is configured to anchor the implanted medical device to tissue of the patient, and wherein the photovoltaic material is located on the fixation mechanism.

Example 28. The method of Example 27, wherein the fixation mechanism comprises one or more of tines, barbs, coils, darts, or hooks.

Example 29. The method of any one of Examples 25 to 28, wherein the photovoltaic material comprises silicon, gallium arsenide, cadmium telluride (CdTe), copper indium gallium selenide (CIGS), or methylammonium lead triiodide (MAPbI₃).

Example 30. The method of any one of Examples 18 to 29, wherein the device component comprises a rechargeable power source.

Example 31. The method of any one of Examples 18 to 30, wherein the device component comprises one or more electrodes configured to sense electrical signals from the patient, deliver electrical stimulation to the patient, or a combination thereof.

Example 32. The method of any one of Examples 18 to 31, wherein the implanted medical device is an insertable cardiac monitor or a leadless pacemaker.

Example 33. The method of any one of Examples 18 to 32, wherein the implanted medical device is configured to be implanted subcutaneously.

### Conclusion

Although many of the embodiments are described above with respect to systems, devices, and methods for cardiac monitoring and/or pacing, the technology is applicable to other applications and/or other approaches, such as other types of implantable medical devices (e.g., defibrillators, cardiac resynchronization pacer/defibrillators, implantable pressure sensors, neurostimulators). Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1-9.

The various processes described herein can be partially or fully implemented using program code including instructions executable by one or more processors of a computing system for implementing specific logical functions or steps in the process. The program code can be stored on any type of computer-readable medium, such as a storage device including a disk or hard drive. Computer-readable media containing code, or portions of code, can include any appropriate media known in the art, such as non-transitory computer-readable storage media. Computer-readable media can include volatile and non-volatile, removable and non-removable media implemented in any method or technology for storage and/or transmission of information, including, but not limited to, random-access memory (RAM), read-only memory (ROM), electrically erasable programmable read-only memory (EEPROM), flash memory, or other memory technology; compact disc read-only memory (CD-ROM), digital video disc (DVD), or other optical storage; magnetic cassettes, magnetic tape, magnetic disk storage, or other magnetic storage devices; solid state drives (SSD) or other solid state storage devices; or any other medium which can be used to store the desired information and which can be accessed by a system device.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded.

To the extent any materials incorporated herein by reference conflict with the present disclosure, the present disclosure controls.

It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1. An implantable medical device comprising:
a housing configured to be implanted in a body of a patient;
an energy harvesting mechanism carried by the housing, the energy harvesting mechanism configured to receive light from a light source located entirely within the body of the patient and to produce electrical energy from the received light; and
electrical circuitry coupled to the energy harvesting mechanism, wherein the electrical circuitry is configured to deliver at least some of the electrical energy to a device component carried by the housing to power the device component.

2. The implantable medical device of claim 1, wherein the light source comprises a tissue of the body of the patient, and wherein the light comprises biophotons emitted by the tissue.

3. The implantable medical device of claim 2, wherein the biophotons comprise a wavelength within a range from 400 nm to 720 nm.

4. The implantable medical device of claim 2, wherein the tissue comprises native cells of the patient that emit the biophotons.

5. The implantable medical device of claim 1, wherein the light source comprises a bioreactor disposed within the housing, the bioreactor comprising a light-emitting species.

6. The implantable medical device of claim 1, wherein the energy harvesting mechanism comprises a photovoltaic material.

7. The implantable medical device of claim 6, wherein the photovoltaic material is located on an external surface of the housing.

8. The implantable medical device of claim 6, wherein the photovoltaic material comprises silicon, gallium arsenide, cadmium telluride (CdTe), copper indium gallium selenide (CIGS), or methylammonium lead triiodide (MAPbI₃).

9. The implantable medical device of claim 1, wherein the device component comprises a rechargeable power source.

10. The implantable medical device of claim 1, wherein the implantable medical device is an insertable cardiac monitor or a leadless pacemaker.

11. A method comprising:
receiving, at a medical device implanted within a body of a patient, light from a light source located entirely within a body of a patient;
producing, via an energy harvesting mechanism of the implanted medical device, electrical energy from the received light; and
powering a device component of the implanted medical device using at least some of the electrical energy.

12. The method of claim 11, wherein the light source comprises a tissue of the body of the patient, and wherein the light comprises biophotons emitted by the tissue.

13. The method of claim 12, wherein the biophotons comprise a wavelength within a range from 400 nm to 720 nm.

14. The method of claim 12, wherein the tissue comprises native cells of the patient that emit the biophotons.

15. The method of claim 11, wherein the light source comprises a bioreactor disposed within the implanted medical device, the bioreactor comprising a light-emitting species.

16. The method of claim 11, wherein the energy harvesting mechanism comprises a photovoltaic material.

17. The method of claim 16, wherein the photovoltaic material is located on an external surface of the implanted medical device.

18. The method of claim 16, wherein the photovoltaic material comprises silicon, gallium arsenide, cadmium telluride (CdTe), copper indium gallium selenide (CIGS), or methylammonium lead triiodide (MAPbI₃).

19. The method of claim 11, wherein the device component comprises a rechargeable power source.

20. The method of claim 11, wherein the implanted medical device is an insertable cardiac monitor or a leadless pacemaker.
